(19) United States
(12) Patent Application Publication
(10) Pub. No.: US 2003/0007956 A1
(43) Pub. Date: Jan. 9, 2003

(54) Proteins that interact with betaTrCP

(76) Inventors: Pierre Legrain, Paris (FR); Richard Benarous, Paris (FR); Guillaume Blot, Sarcelles (FR); Irina Lassot, Sarcelles (FR)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/023,530

(22) Filed: Dec. 18, 2001

Related U.S. Application Data
(60) Provisional application No. 60/256,276, filed on Dec. 18, 2000.

(51) Int. Cl. A61K 48/00; C12P 21/02; C12N 5/06; C12N 9/00

(52) U.S. Cl. 424/93.21; 435/183; 435/320.1; 435/325; 435/455; 435/69.1

(57) ABSTRACT

The present invention relates to proteins that interaction with βTrCP. More specifically, the present invention relates to complexes of polypeptides or polynucleotides encoding the polypeptides, fragments of the polypeptides, antibodies to the complexes, Selected Interacting Domains (SID®) which are identified due to the protein-protein interactions, methods for screening drugs for agents which modulate the interaction of proteins and pharmaceutical compositions that are capable of modulating the protein-protein interactions.

### FIELD OF THE INVENTION

The present invention relates to proteins that interact with βTrCP such as RasSF1, RasSF1A and RasSF1C and variants of RasSF1. More specifically, the present invention relates to complexes of polypeptides or polynucleotides encoding the polypeptides, fragments of the polypeptides, antibodies to the complexes, Selected Interacting Domains (SID®) which are identified due to the protein-protein interactions, methods for screening drugs for agents which modulate the interaction of proteins and pharmaceutical compositions that are capable of modulating the protein-protein interactions.

### BACKGROUND AND PRIOR ART

Most biological processes involve specific protein-protein interactions. Protein-protein interactions enable two or more proteins to associate. A large number of non-covalent bonds form between the proteins when two protein surfaces are precisely matched. These bonds account for the specificity of recognition. Thus, protein-protein interactions are involved, for example, in the assembly of enzyme subunits, in antibody-antigen recognition, in the formation of biochemical complexes, in the correct folding of proteins, in the metabolism of proteins, in the transport of proteins, in the localization of proteins, in protein turnover, in first translation modifications, in the core structures of viruses and in signal transduction.

General methodologies to identify interacting proteins or to study these interactions have been developed. Among these methods are the two-hybrid system originally developed by Fields and co-workers and described, for example, in U.S. Pat. Nos. 5,283,173, 5,468,614 and 5,667,973, which are hereby incorporated by reference.

The earliest and simplest two-hybrid system, which acted as basis for development of other versions, is an in vivo assay between two specifically constructed proteins. The first protein, known in the art as the “bait protein” is a chimeric protein which binds to a site on DNA upstream of a reporter gene by means of a DNA-binding domain or BD. Commonly, the binding domain is the DNA-binding domain from either Gal4 or native E. coli LexA and the sites placed upstream of the reporter are Gal4 binding sites or LexA operators, respectively.

The second protein is also a chimeric protein known as the “prey” in the art. This second chimeric protein carries an activation domain or AD. This activation domain is typically derived from Gal4, from VP16 or from B42.

Besides the two hybrid systems, other improved systems have been developed to detected protein-protein interactions. For example, a two-hybrid plus one system was developed that allows the use of two proteins as bait to screen available cDNA libraries to detect a third partner. This method permits the detection between proteins that are part of a larger protein complex such as the RNA polymerase II holoenzyme and the TFIIH or TFIID complexes. Therefore, this method, in general, permits the detection of ternary complex formation as well as inhibitors preventing the interaction between the two previously defined fused proteins.

Another advantage of the two-hybrid plus one system is that it allows or prevents the formation of the transcriptional activator since the third partner can be expressed from a conditional promoter such as the methionine-repressed Met25 promoter which is positively regulated in medium lacking methionine. The presence of the methionine-regulated promoter provides an excellent control to evaluate the activation or inhibition properties of the third partner due to its “on” and “off” switch for the formation of the transcriptional activator. The three-hybrid method is described, for example in Tirode et al., The Journal of Biological Chemistry, 272, No. 37 pp. 22995-22999 (1997). incorporated herein by reference.

Besides the two and two-hybrid plus one systems, yet another variant is that described in Vidal et al, Proc. Natl. Sci. 93 pgs. 10315-10320 called the reverse two- and one-hybrid systems where a collection of molecules can be screened that inhibit specific protein-protein or protein/DNA interactions, respectively.

A summary of the available methodologies for detecting protein-protein interactions is described in Vidal and Legrain, Nucleic Acids Research Vol. 27, No.4 pgs.919-929 (1999) and Legrain and Selig, FEBS Letters, 480 pgs. 32-36 (2000) which references are incorporated herein by reference.

However, the above conventionally used approaches and especially the commonly used two-hybrid methods have their drawbacks. For example, it is known in the art that, more often than not, false positives and false negatives exist in the screening method. In fact, a doctrine has been developed in this field for interpreting the results and in common practice an additional technique such as co-immunoprecipitation or gradient sedimentation of the putative interactors from the appropriate cell or tissue type are generally performed. The methods used for interpreting the results are described by Brent and Finley, Jr. in Ann. Rev. Genet., 31 pgs. 663-704 (1997). Thus, the data interpretation is very questionable using the conventional systems.

One method to overcome the difficulties encountered with the methods in the prior art is described in WO 99/42612, incorporated herein by reference. This method is similar to the two-hybrid system described in the prior art in that it also uses bait and prey polypeptides. However, the difference with this method is that a step of mating at least one first haploid recombinant yeast cell containing the prey polypeptide to be assayed with a second haploid recombinant yeast cell containing the bait polynucleotide is performed. Of course the person skilled in the art would appreciate that either the first recombinant yeast cell or the second recombinant yeast cell also contains at least one detectable reporter gene that is activated by a polypeptide including a transcriptional activation domain.

The method described in WO 99/42612 permits the screening of more prey polynucleotides with a given bait polynucleotide in a single step than in the prior art systems due to the cell to cell mating strategy between haploid yeast cells. Furthermore, this method is more thorough and reproducible, as well as sensitive. Thus, the presence of false negatives and/or false positives is extremely minimal as compared to the conventional prior art methods.

F-box proteins are members of a large family that regulates the cell cycle, the immune response, signalling cascades and developmental programs by targeting proteins for ubiquitination and thus subsequent degradation by the 26S proteasome (Patton et al. Trends Genet., 14, 236-243 (1998); Koepp et al., Cell, 97, 431-434(1999); Deshaies et al., Annu. Rev. Cell Dev. Biol. 15, 425-467 (1999)). F-box proteins are the substrate recognition components of SCF (Skpl-Cullin-F-box protein) ubiquitin ligases (Skowrya et al., Cell, 91, 209-219 (1997); Feldman et al., Cell, 91, 221-230 (1997)). They bind the SCF constant catalytic core by means of the F-box motif interacting with Skpl, and they bind substrates through their variable protein-protein interaction domains (Bai et al., 1996). One of these, βTrCP is a protein implicated in the regulation of the degradation of proteins phosphorylated upon two Serine residues present in the motif DSGXXS (IκB, β-catenin). βTrCP can be divided into two domains: an N-terminal domain which contains the F-box motif (InterPro001810, 154-192 amino acids) and a C-terminal domain, containing 7 “WD40 repeats” (InterPro001680) which are responsible for binding to the DSGXXS motif in the proteins to be ubiquitinated and subsequently degraded. As well as the cellular substrates described above, βTrCP also binds to the Vpu protein of HIV-1 via a DSGXXS motif present in the viral protein (Margottin et al., Mol Cell., 1, 565-74 (1998)). The first three WD40 repeat motifs are sufficient for the interaction βTrCP/Vpu to be detectable in a yeast two hybrid assay (Margottin et al., supra)

RasSF1 is a protein that was originally found as a protein that interacts in a yeast two-hybrid assay with XPA, a protein that is implicated in DNA repair. The gene encoding the human RasSF1 protein is located at the 3p21.3 locus (Dammann et al., Nature Genetics, 25, pgs. 315-319 (2000), a region implicated in tumor suppression and frequently observed to be homozygous in lung cancers (both small cell and non-small cell lung carcinomas). The gene for RasSF1 encodes for many protein isoforms, two of which (RasSF1A and RasSF1C) contain the same Ras-association domain (InterPro000159) and have been shown to be absent in tumor cell lines (Dammann et al., supra); Vos et al., J. Biol. Chem, 275, 35669-35672(2000)). Furthermore, ectopic expression of RasSF1A inhibits the tumor-forming potential in nude mice of such cell cells (Dammann et al., supra). RasSF1A and RasSF1C are considered to be potential tumor suppressor proteins.

Thus, it is an object of the present invention to identify proteins that interact with βTrCP such as RasSF1 and variants thereof.

It is another object of the present invention to identify protein-protein interactions of βTrCP with RasSF1 and variants of RasSF1 for the development of more effective and better targeted therapeutic applications.

It is another object of the present invention to identify specific domains on RasSF1 that interact with βTrCP and specific domains on βTrCP that interact with RasSF1.

It is yet another object of the present invention to identify complexes of polypeptides or polynucleotides encoding the polypeptides and fragments of the polypeptides of βTrCP with RasSF1.

It is yet another object of the present invention to identify antibodies to these complexes of polypeptides or polynucleotides encoding the polypeptides and fragments of the polypeptides of βTrCP with RasSF1 including polyclonal, as well as monoclonal antibodies that are used for detection.

It is still another object of the present invention to identify selected interacting domains of the polypeptides, called SID® polypeptides.

It is still another object of the present invention to identify selected interacting domains of the polynucleotides, called SID® polynucleotides.

It is yet another object of the present invention to provide a method for screening drugs for agents, which modulate the interaction of proteins, and pharmaceutical compositions that are capable of modulating the protein-protein interactions of βTrCP with RasSF1.

It is yet another object of the present invention to provide protein chips or protein microarrays.

It is yet another object of he present invention to provide a report in, for example paper, electronic and/or digital forms, concerning the protein-protein interactions, the modulating compounds and the like.

These and other objects are achieved by the present invention as evidenced by the summary of the invention, description of the preferred embodiments and the claims.

### SUMMARY OF THE PRESENT INVENTION

Thus the present invention relates to a protein complex of βTrCP and RasSF1 and variants of RasSF1.

In another embodiment the present invention provides a method for screening drugs for agents that modulate the protein-protein interactions of βTrCP and RasSF1 and variants of RasSF1, and pharmaceutical compositions that are capable of modulating protein-protein interactions.

In another embodiment the present invention provides protein microarrays of βTrCP and RasSF1 and variants of RasSF1.

In yet another embodiment the present invention provides a report in, for example, paper, electronic and/or digital forms.

### BRIEF DESCRIPTION OF THE DRAWINGS

FIG. 1 is a schematic representation of the pB1 plasmid.

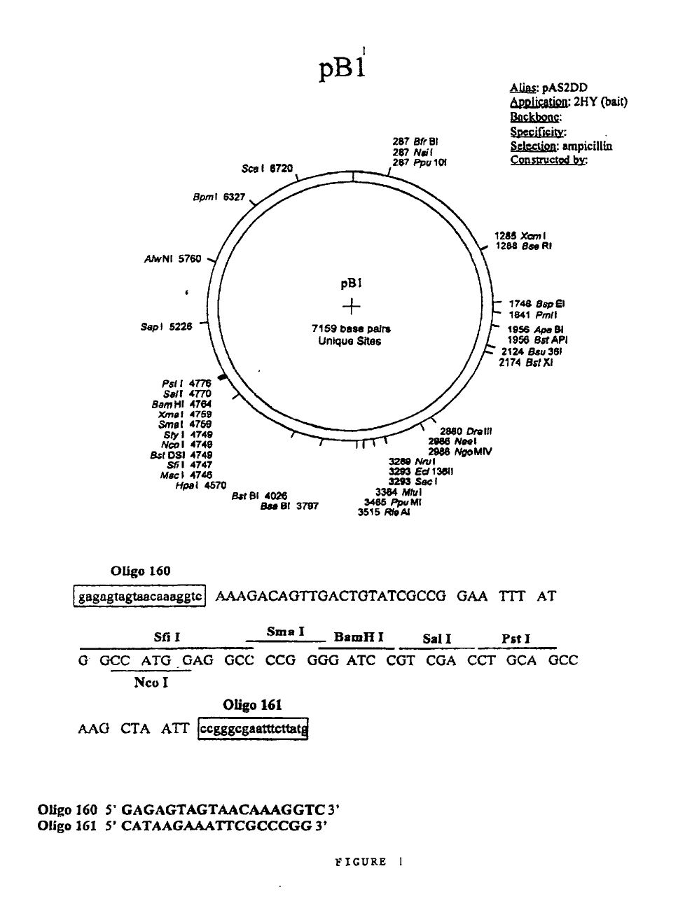

FIG. 2 is a schematic representation of the pB5 plasmid.

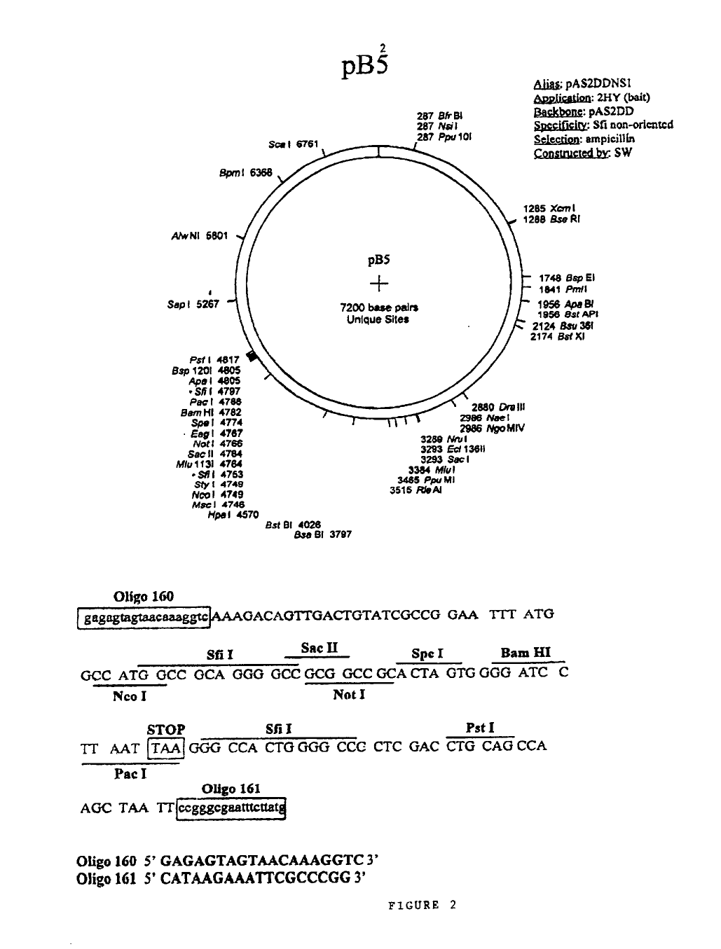

FIG. 3 is a schematic representation of the pB6 plasmid.

FIG. 4 is a schematic representation of the pB13 plasmid.

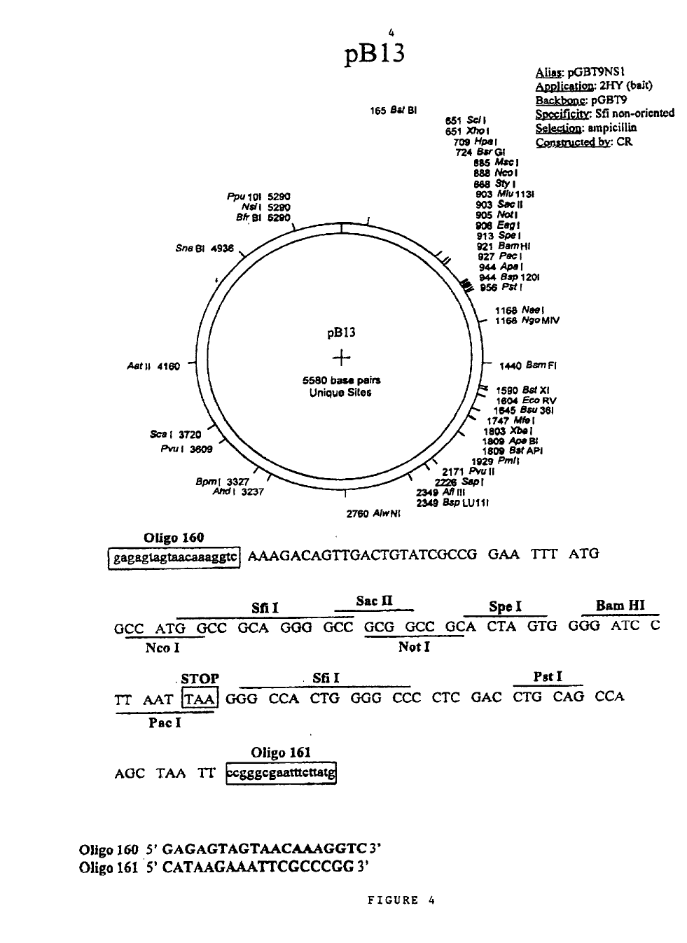

FIG. 5 is a schematic representation of the pB14 plasmid.

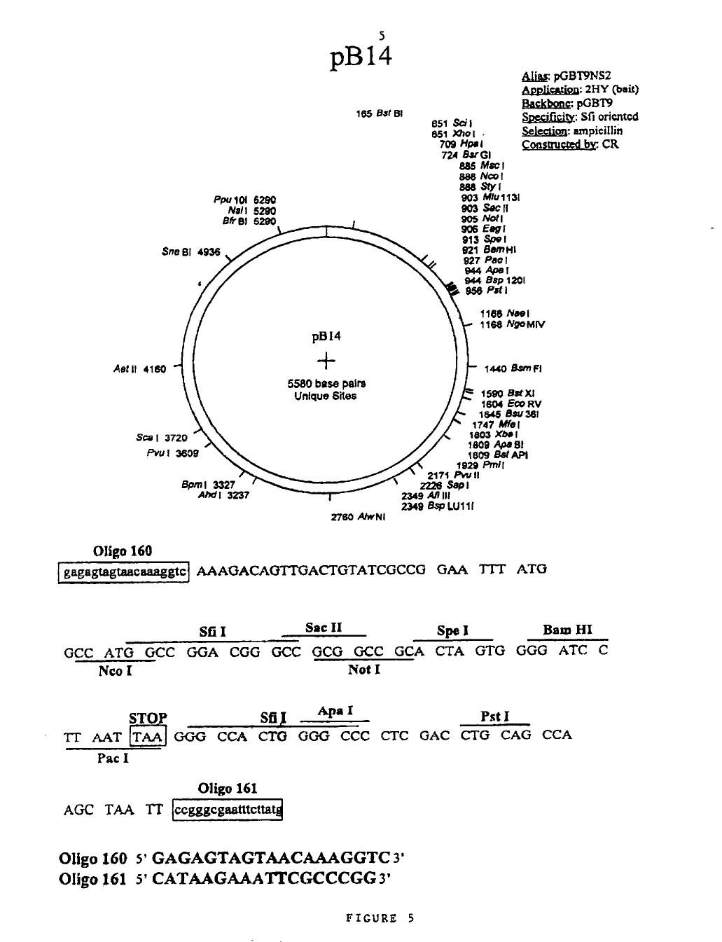

FIG. 6 is a schematic representation of the pB20 plasmid.

FIG. 7 is a schematic representation of the pP1 plasmid.

FIG. 8 is a schematic representation of the pP2 plasmid.

FIG. 9 is a schematic representation of the pP3 plasmid.

FIG. 10 is a schematic representation of the pP6 plasmid.

FIG. 11 is a schematic representation of the pP7 plasmid.

FIG. 12 is a schematic representation of vectors expressing the T25 fragment.

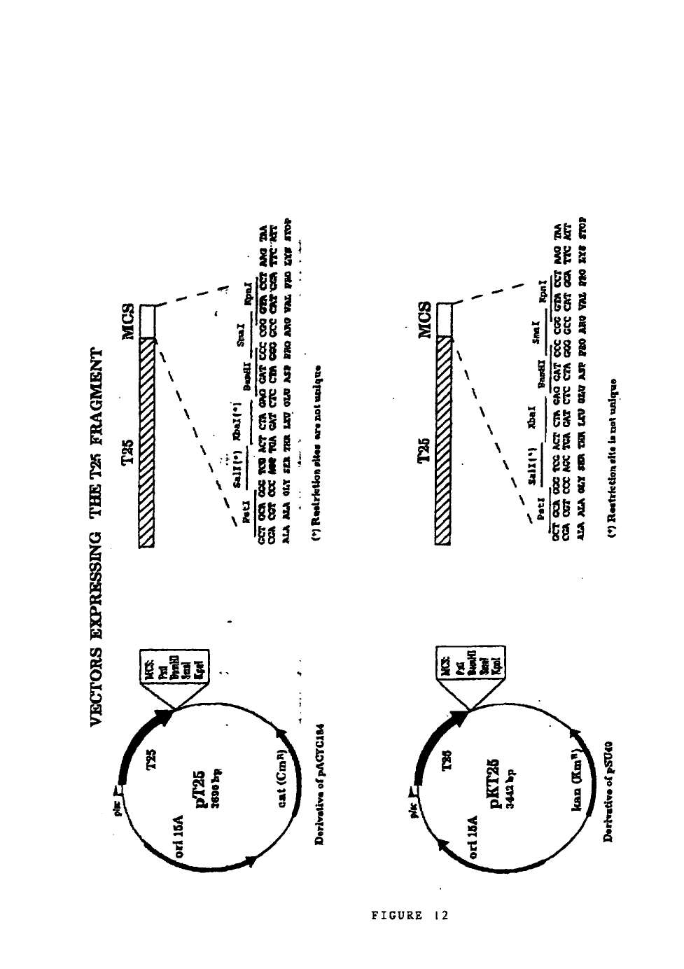

FIG. 13 is a schematic representation of vectors expressing the T18 fragment.

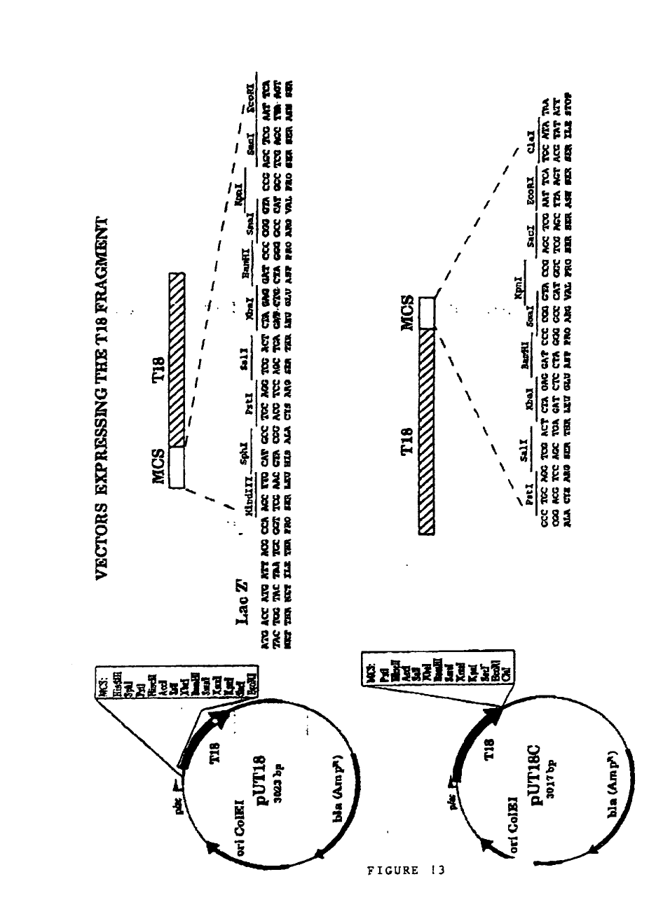

FIG. 14 is a schematic representation of various vectors of pCmAHL1, pT25 and pT18.

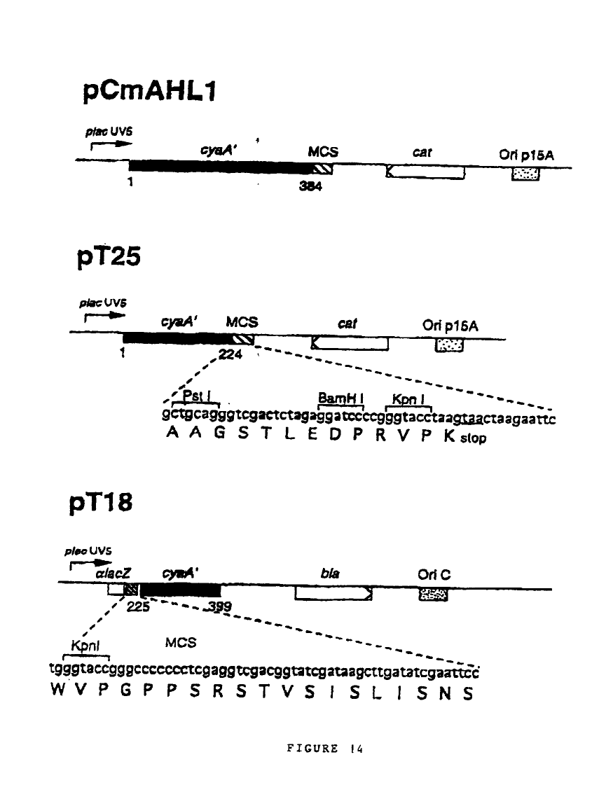

FIG. 15 are Western blots of RasSF1A and Ras SF1C co-immunoprecipitated with β-TrCP in the human cell line 293.

FIG. 16 are Western blots illustrating that RasSF1C can immunoprecipitate with β-TrCPΔN (having amino acids 1 to 143 of β-TrCP deleted) or with β-TrCPΔF (having amino acids 32 to 179 of β-TrCP deleted).

FIG. 17 are Western blots of various deletion mutants of β-TrCP illustrating that RasSF1C can immunoprecipitate with β-TrCP 1-333 but not with β-TrCP 1-260 or with -TrCP 261-569.

FIG. 18 are SDS gels illustrating that HA-β-TrCP is co-immunoprecipitated with Myc-RasSF1A using an anti-myc immunoprecipitate and hence confirms that RasSF1 and β-TrCP are associated in human cells.

FIG. 19 are Western blots illustrating that inhibition of RasSF1 expression by RNAi targeting RasSF1 mRNA, results in a decrease of β-catenin expression, while overexpression of RasSF1 results in an increase of β-catenin expression.

FIG. 20 is a diagram illustrating the domain specifically required on βTrCP for interaction with RasSF1.

### DETAILED DESCRIPTION OF THE PREFERRED EMBODIMENTS

As used herein the terms “polynucleotides,” “nucleic acids,” and “oligonucleotides” are used interchangeably and include, but are not limited to RNA, DNA, RNA/DNA sequences of more than one nucleotide in either single chain or duplex form. The polynucleotide sequences of the present invention may be prepared from any known method including, but not limited to, any synthetic method, any recombinant method, any ex vivo generation method and the like, as well as combinations thereof.

The term “polypeptide” means herein a polymer of amino acids having no specific length. Thus, peptides, oligopeptides and proteins are included in the definition of “polypeptide” and these terms are used interchangeably throughout the specification, as well as in the claims. The term “polypeptide” does not exclude post-translational modifications such as polypeptides having covalent attachment of glycosyl groups, acetyl groups, phosphate groups, lipid groups and the like. Also encompassed by this definition of “polypeptide” are homologs thereof.

By the term “homologs” is meant structurally similar genes contained within a given species, orthologs are functionally equivalent genes from a given species or strain, as determined for example, in a standard complementation assay. Thus, a polypeptide of interest can be used not only as a model for identifying similar genes in given strains, but also to identify homologs and orthologs of the polypeptide of interest in other species. The orthologs, for example, can also be identified in a conventional complementation assay. In addition or alternatively, such orthologs can be expected to exist in bacteria (or other kind of cells) in the same branch of the phylogenic tree, as set forth, for example, at ftp://ftp.cme.msu.edu/pub/rdp/SSU-rRNA/SSU/Prok.phylo.

As used herein the term “prey polynucleotide” means a chimeric polynucleotide encoding a polypeptide comprising (i) a specific domain; and (ii) a polypeptide that is to be tested for interaction with a bait polypeptide. The specific domain is preferably a transcriptional activating domain.

As used herein, a “bait polynucleotide” is a chimeric polynucleotide encoding a chimeric polypeptide comprising (i) a complementary domain; and (ii) a polypeptide that is to be tested for interaction with at least one prey polypeptide. The complementary domain is preferably a DNA-binding domain that recognizes a binding site that is further detected and is contained in the host organism.

As used herein “complementary domain” is meant a functional constitution of the activity when bait and prey are interacting; for example, enzymatic activity.

As used herein “specific domain” is meant a functional interacting activation domain that may work through different mechanisms by interacting directly or indirectly through intermediary proteins with RNA polymerase II or III-associated proteins in the vicinity of the transcription start site.

As used herein the term “complementary” means that, for example, each base of a first polynucleotide is paired with the complementary base of a second polynucleotide whose orientation is reversed. The complementary bases are A and T (or A and U) or C and G.

The term “sequence identity” refers to the identity between two peptides or between two nucleic acids. Identity between sequences can be determined by comparing a position in each of the sequences which may be aligned for the purposes of comparison. When a position in the compared sequences is occupied by the same base or amino acid, then the sequences are identical at that position. A degree of sequence identity between nucleic acid sequences is a function of the number of identical nucleotides at positions shared by these sequences. A degree of identity between amino acid sequences is a function of the number of identical amino acid sequences that are shared between these sequences. Since two polypeptides may each (i) comprise a sequence (i.e., a portion of a complete polynucleotide sequence) that is similar between two polynucleotides, and (ii) may further comprise a sequence that is divergent between two polynucleotides, sequence identity comparisons between two or more polynucleotides over a “comparison window” refers to the conceptual segment of at least 20 contiguous nucleotide positions wherein a polynucleotide sequence may be compared to a reference nucleotide sequence of at least 20 contiguous nucleotides and wherein the portion of the polynucleotide sequence in the comparison window may comprise additions or deletions (i.e., gaps) of 20 percent or less compared to the reference sequence (which does not comprise additions or deletions) for optimal alignment of the two sequences.

To determine the percent identity of two amino acids sequences or two nucleic acid sequences, the sequences are aligned for optimal comparison. For example, gaps can be introduced in the sequence of a first amino acid sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position.

The percent identity between the two sequences is a function of the number of identical positions shared by the sequences. Hence % identity=number of identical positions/total number of overlapping positions×100.

In this comparison the sequences can be the same length or may be different in length. Optimal alignment of sequences for determining a comparison window may be conducted by the local homology algorithm of Smith and Waterman (J. Theor. Biol. 91(2) pgs. 379-380 (1981), by the homology alignment algorithm of Needleman and Wunsch, J. Mol. Biol., 48(3) pgs. 443-453 (1972), by the search for similarity via the method of Pearson and Lipman, PNAS, USA, 85(8) pgs. 2444-2448 (1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection.

The best alignment (i.e., resulting in the highest percentage of identity over the comparison window) generated by the various methods is selected.

The term “sequence identity” means that two polynucleotide sequences are identical (i.e., on a nucleotide by nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or 1) occurs in both sequences to yield the number of matched positions, dividing the number of matched positions by the total number of positions in the window of comparison (i.e., the window size) and multiplying the result by 100 to yield the percentage of sequence identity. The same process can be applied to polypeptide sequences.

The percentage of sequence identity of a nucleic acid sequence or an amino acid sequence can also be calculated using BLAST software (Version 2.06 of September 1998) with the default or user defined parameter.

The term “sequence similarity” means that amino acids can be modified while retaining the same function. It is known that amino acids are classified according to the nature of their side groups and some amino acids such as the basic amino acids can be interchanged for one another while their basic function is maintained.

The term “isolated” as used herein means that a biological material such as a nucleic acid or protein has been removed from its original environment in which it is naturally present. For example, a polynucleotide present in a plant, mammal or animal is present in its natural state and is not considered to be isolated. The same polynucleotide separated from the adjacent nucleic acid sequences in which it is naturally inserted in the genome of the plant or animal is considered as being “isolated.”

The term “isolated” is not meant to exclude artificial or synthetic mixtures with other compounds, or the presence of impurities which do not interfere with the biological activity and which may be present, for example, due to incomplete purification, addition of stabilizers or mixtures with pharmaceutically acceptable excipients and the like.

“Isolated polypeptide” or “isolated protein” as used herein means a polypeptide or protein which is substantially free of those compounds that are normally associated with the polypeptide or protein in a naturally state such as other proteins or polypeptides, nucleic acids, carbohydrates, lipids and the like.

The term “purified” as used herein means at least one order of magnitude of purification is achieved, preferably two or three orders of magnitude, most preferably four or five orders of magnitude of purification of the starting material or of the natural material. Thus, the term “purified” as utilized herein does not mean that the material is 100% purified and thus excludes any other material.

The term “variants” when referring to, for example, polynucleotides encoding a polypeptide variant of a given reference polypeptide are polynucleotides that differ from the reference polypeptide but generally maintain their functional characteristics of the reference polypeptide. A variant of a polynucleotide may be a naturally occurring allelic variant or it may be a variant that is known naturally not to occur. Such non-naturally occurring variants of the reference polynucleotide can be made by, for example, mutagenesis techniques, including those mutagenesis techniques that are applied to polynucleotides, cells or organisms.

Generally, differences are limited so that the nucleotide sequences of the reference and variant are closely similar overall and, in many regions identical.

Variants of polynucleotides according to the present invention include, but are not limited to, nucleotide sequences which are at least 95% identical after alignment to the reference polynucleotide encoding the reference polypeptide. These variants can also have 96%, 97%, 98% and 99.999% sequence identity to the reference polynucleotide.

Nucleotide changes present in a variant polynucleotide may be silent, which means that these changes do not alter the amino acid sequences encoded by the reference polynucleotide.

Substitutions, additions and/or deletions can involve one or more nucleic acids. Alterations can produce conservative or non-conservative amino acid substitutions, deletions and/or additions.

Variants of a prey or a SID® polypeptide encoded by a variant polynucleotide can possess a higher affinity of binding and/or a higher specificity of binding to its protein or polypeptide counterpart, against which it has been initially selected. In another context, variants can also loose their ability to bind to their protein or polypeptide counterpart.

By “anabolic pathway” is meant a reaction or series of reactions in a metabolic pathway that synthesize complex molecules from simpler ones, usually requiring the input of energy. An anabolic pathway is the opposite of a catabolic pathway.

As used herein, a “catabolic pathway” is a series of reactions in a metabolic pathway that break down complex compounds into simpler ones, usually releasing energy in the process. A catabolic pathway is the opposite of an anabolic pathway.

As used herein, “drug metabolism” is meant the study of how drugs are processed and broken down by the body. Drug metabolism can involve the study of enzymes that break down drugs, the study of how different drugs interact within the body and how diet and other ingested compounds affect the way the body processes drugs.

As used herein, “metabolism” means the sum of all of the enzyme-catalyzed reactions in living cells that transform organic molecules.

By “secondary metabolism” is meant pathways producing specialized metabolic products that are not found in every cell.

As used herein, “SID®” means a Selected Interacting Domain and is identified as follows: for each bait polypeptide screened, selected prey polypeptides are compared. Overlapping fragments in the same ORF or CDS define the selected interacting domain.

As used herein the term “PIM®” means a protein-protein interaction map. This map is obtained from data acquired from a number of separate screens using different bait polypeptides and is designed to map out all of the interactions between the polypeptides.

The term “affinity of binding”, as used herein, can be defined as the affinity constant Ka when a given SID® polypeptide of the present invention which binds to a polypeptide and is the following mathematical relationship:

$$Ka = \frac{[ SID ® / polypeptide complex ]}{[ free SID ® ] [ free polypeptide ]}$$

wherein [free SID®], [free polypeptide] and [SID®/polypeptide complex] consist of the concentrations at equilibrium respectively of the free SID® polypeptide, of the free polypeptide onto which the SID® polypeptide binds and of the complex formed between SID® polypeptide and the polypeptide onto which said SID® polypeptide specifically binds.

The affinity of a SID® polypeptide of the present invention or a variant thereof for its polypeptide counterpart can be assessed, for example, on a Biacore™ apparatus marketed by Amersham Pharmacia Biotech Company such as described by Szabo et al Curr Opin Struct Biol 5 pgs. 699-705 (1995) and by Edwards and Leartherbarrow, Anal. Biochem 246 pgs. 1-6 (1997).

As used herein the phrase “at least the same affinity” with respect to the binding affinity between a SID® polypeptide of the present invention to another polypeptide means that the Ka is identical or can be at least two-fold, at least three-fold or at least five fold greater than the Ka value of reference.

As used herein, the term “modulating compound” means a compound that inhibits or stimulates or can act on another protein which can inhibit or stimulate the protein-protein interaction of a complex of two polypeptides or the protein-protein interaction of two polypeptides.

More specifically, the present invention comprises complexes of polypeptides or polynucleotides encoding the polypeptides composed of a bait polypeptide, or a bait polynucleotide encoding a bait polypeptide and a prey polypeptide or a prey polynucleotide encoding a prey polypeptide. The prey polypeptide or prey polynucleotide encoding the prey polypeptide is capable of interacting with a bait polypeptide of interest in various hybrid systems.

As described in the Background of the present invention there are various methods known in the art to identify prey polypeptides that interact with bait polypeptides of interest. These methods, include, but are not limited to, generic two-hybrid systems as described by Fields et al in Nature, 340:245-246 (1989) and more specifically in U.S. Pat. Nos. 5,283,173, 5,468,614 and 5,667,973, which are hereby incorporated by reference; the reverse two-hybrid system described by Vidal et al, supra; the two plus one hybrid method described, for example, in Tirode et al, supra; the yeast forward and reverse ‘n’-hybrid systems as described in Vidal and Legrain, supra; the method described in WO 99/42612; those methods described in Legrain et al FEBS Letters 480 pgs. 32-36 (2000) and the like.

The present invention is not limited to the type of method utilized to detect protein-protein interactions and therefore any method known in the art and variants thereof can be used. It is however better to use the method described in WO 99/42612 or WO 00/66722, both references incorporated herein by reference, due to the methods' sensitivity, reproducibility and reliability.

Protein-protein interactions can also be detected using complementation assays such as those described by Pelltier et al at http://www.abrf.org/JBT/Articles/JBT0012/jbt0012.html, WO 00/07038 and WO98/34120.

Although the above methods are described for applications in the yeast system, the present invention is not limited to detecting protein-protein interactions using yeast, but also includes similar methods that can be used in detecting protein-protein interactions in, for example, mammalian systems as described, for example in Takacs et al., Proc. Natl. Acad. Sci., USA, 90 (21):10375-79 (1993) and Vasavada et al., Proc. Nati. Acad. Sci., USA, 88 (23):10686-90 (1991), as well as a bacterial two-hybrid system as described in Karimova et al (1998), WO99/28746, WO 00/66722 and Legrain et al FEBS Letters, 480 pgs. 32-36 (2000).

The above-described methods are limited to the use of yeast, mammalian cells and Escherichia coli cells, the present invention is not limited in this manner. Consequently, mammalian and typically human cells, as well as bacterial, yeast, fungus, insect, nematode and plant cells are encompassed by the present invention and may be transfected by the nucleic acid or recombinant vector as defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli, (e.g., strain DH5-α), Bacillus subtilis, Salmonella typhimurium, or strains of the genera of Pseudomonas, Streptomyces and Staphylococcus.

Further suitable cells that can be used in the present invention include yeast cells such as those of Saccharomyces such as Saccharomyces cerevisiae.

The bait polynucleotide, as well as the prey polynucleotide can be prepared according to the methods known in the art such as those described above in the publications and patents reciting the known method per se.

The bait polynucleotide of the present invention is βTrCP. The prey polynucleotide is RasSF1, variants of RasSF1 and fragments from the genome or transcriptome of RasSF1 ranging from about 12 to about 2,000. The prey polynucleotide is then selected, sequenced and identified.

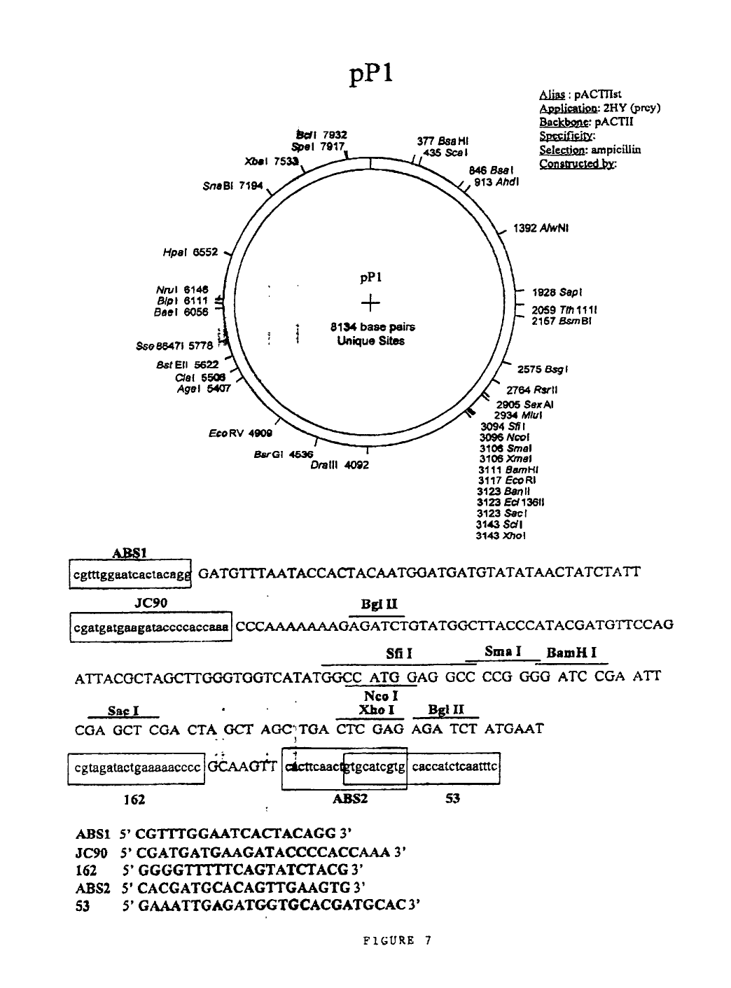

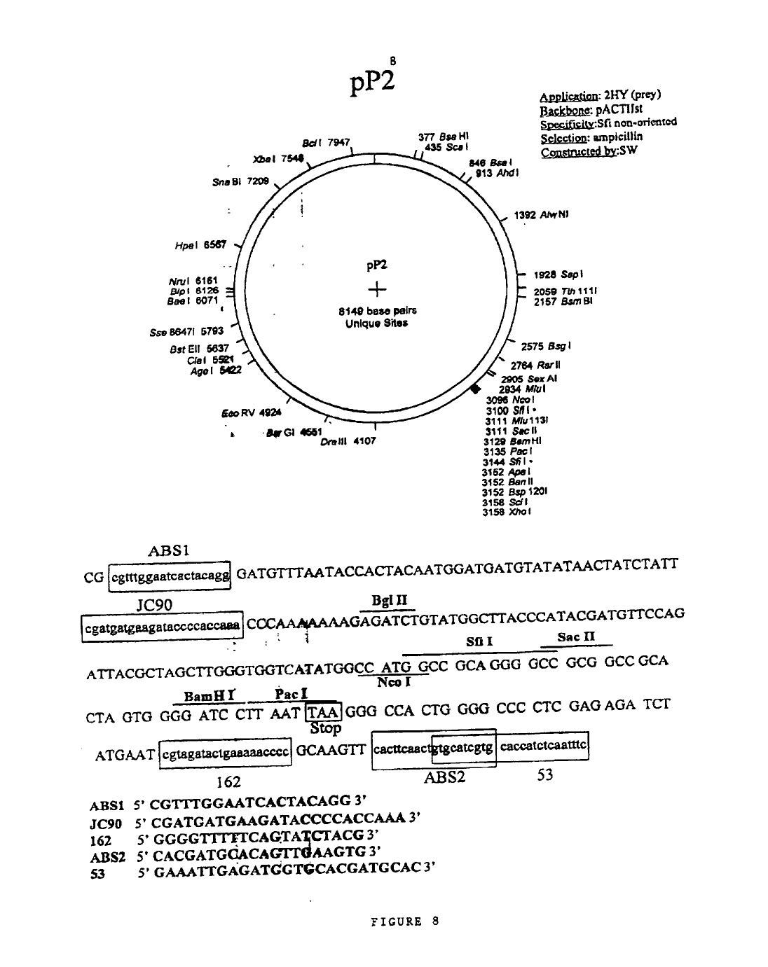

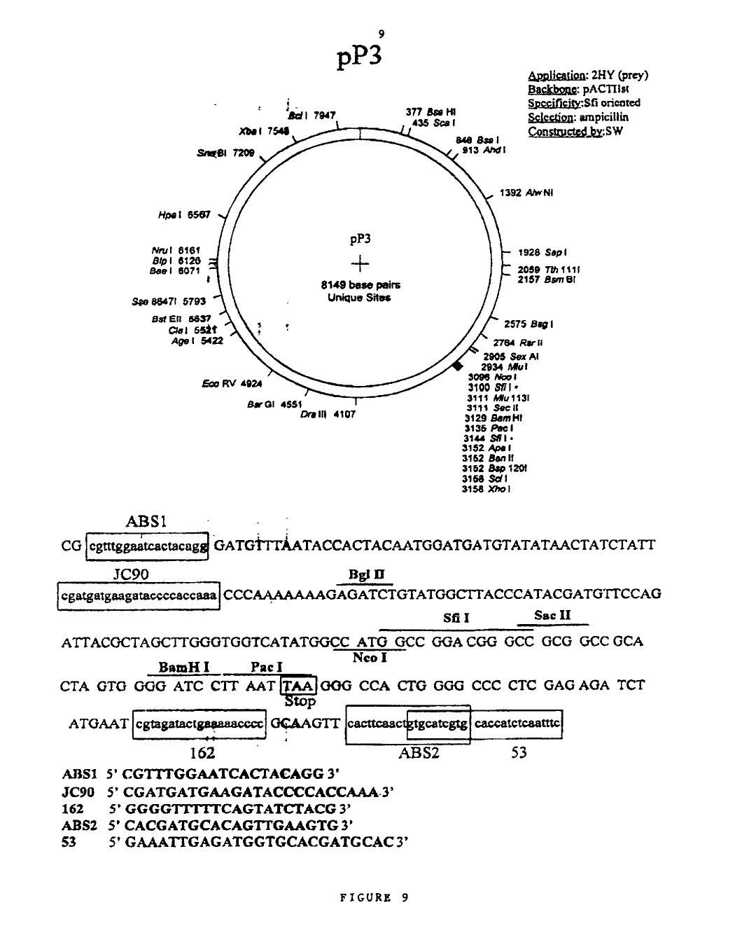

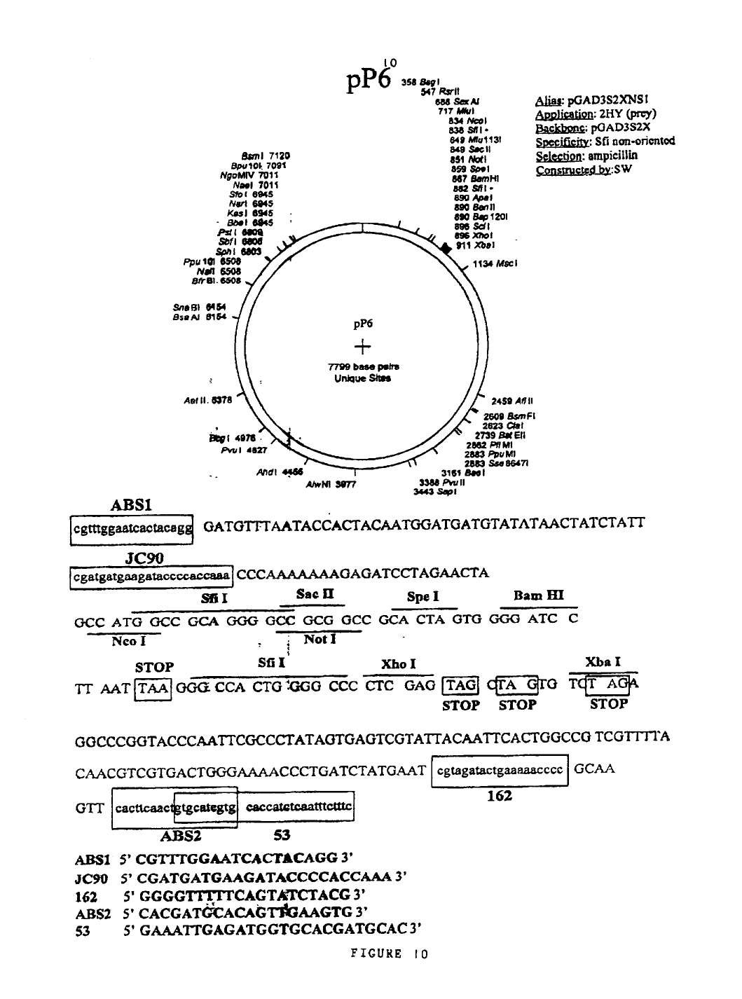

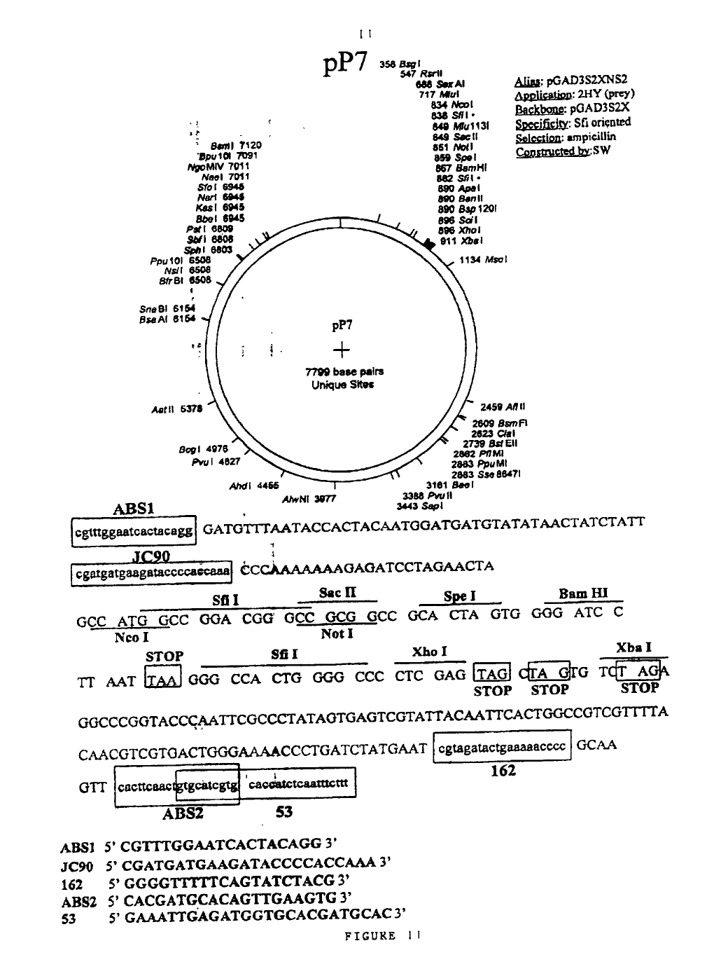

A prey library is prepared from human placenta, human undifferentiated PAZ6 adipocytes and human differentiated PAZ6 adipocytes and constructed in the specially designed prey vector pP6 as shown in FIG. 10 after ligation of suitable linkers such that every RasSF1 insert is fused to a nucleotide sequence in the vector that encodes the transcription activation domain of a reporter gene. The present invention is not limited to the use of the prey vector pP6. Any vector in FIGS. 7 to 11 can be used. Any transcription activation domain can be used in the present invention. Examples include, but are not limited to, Gal4,YP16, B42, His and the like. Toxic reporter genes, such as CAT^{R}, CYH2, CYHI, URA3, bacterial and fungi toxins and the like can be used in reverse two-hybrid systems.

The polypeptides encoded by the nucleotide inserts of the RasSF1 prey library thus prepared are termed “prey polypeptides” in the context of the presently described selection method of the prey polynucleotides.

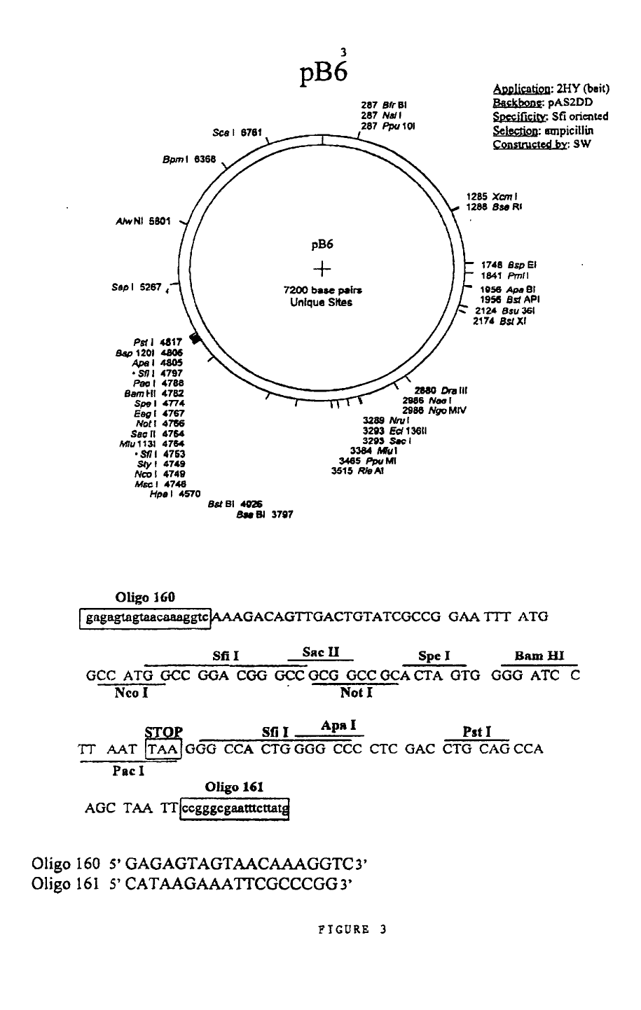

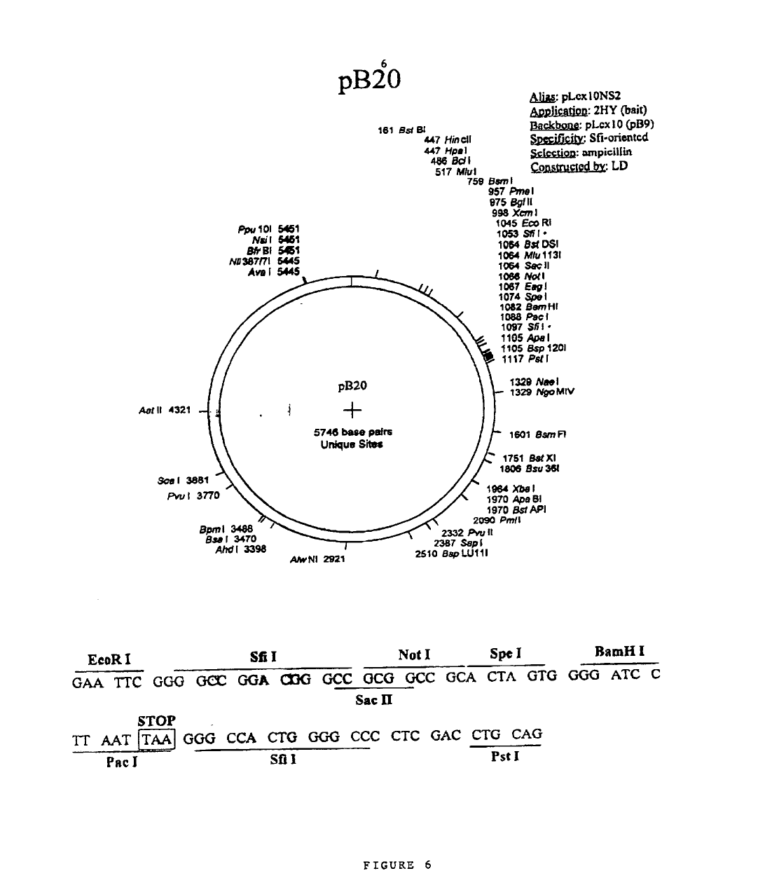

The bait polynucleotide can be inserted in bait plasmid as illustrated in FIGS. 3 and 6. The bait polynucleotide insert is fused to a polynucleotide encoding the binding domain of, for example, the Gal4 DNA binding domain and the shuttle expression vector is used to transform cells.

As stated above, any cells can be utilized in transforming the bait and prey polynucleotides of the present invention including mammalian cells, bacterial cells, yeast cells, insect cells and the like.

In an embodiment, the present invention identifies protein-protein interactions in yeast. In using known methods a prey positive clone is identified containing a vector which comprises a nucleic acid insert encoding a prey polypeptide which binds to a bait polypeptide of interest. The method in which protein-protein interactions are identified comprises the following steps:

i) mating at least one first haploid recombinant yeast cell clone from a recombinant yeast cell clone library that has been transformed with a plasmid containing the prey polynucleotide to be assayed with a second haploid recombinant yeast cell clone transformed with a plasmid containing a bait polynucleotide encoding for the bait polypeptide;

ii) cultivating diploid cell clones obtained in step i) on a selective medium; and

iii) selecting recombinant cell clones which grow on the selective medium.

This method may further comprise the step of:

iv) characterizing the prey polynucleotide contained in each recombinant cell clone which is selected in step iii).

In yet another embodiment of the present invention, in lieu of yeast, Escherichia coli is used in a bacterial two-hybrid system, which encompasses a similar principle to that described above for yeast, but does not involve mating for characterizing the prey polynucleotide.

In yet another embodiment of the present invention, mammalian cells and a method similar to that described above for yeast for characterizing the prey polynucleotide are used.

By performing the yeast, bacterial or mammalian two-hybrid system it is possible to identify for one particular bait an interacting prey polypeptide. The prey polypeptide that has been selected by testing the library of preys in a screen using the two-hybrid, two plus one hybrid methods and the like, encodes the polypeptide interacting with the protein of interest.

The present invention is also directed, in a general aspect, to a complex of polypeptides, polynucleotides encoding the polypeptides composed of a bait polypeptide or bait polynucleotide encoding the bait polypeptide and a prey polypeptide or prey polynucleotide encoding the prey polypeptide capable of interacting with the bait polypeptide of interest. These complexes are identified in as the bait amino acid sequences and the prey amino acid sequences in SEQ ID Nos. 2 and 4, as well as the bait and prey nucleic acid sequences, in SEQ ID Nos. 1 and 3.

In another aspect, the present invention relates to a complex of polynucleotides consisting of a first polynucleotide, or a fragment thereof, encoding a prey polypeptide that interacts with a bait polypeptide and a second polynucleotide or a fragment thereof. This fragment has at least 12 consecutive nucleotides, but can have between 12 and 5,000 consecutive nucleotides, or between 12 and 10,000 consecutive nucleotides or between 12 and 20,000 consecutive nucleotides.

The polypeptides (SEQ ID Nos. 2 and 4) encoded by the polynucleotides (SEQ ID Nos. 1 and 3) according to the present invention and the complexes of the two polypeptides encoded by the sets of two polynucleotides also form part of the present invention.

In yet another embodiment, the present invention relates to an isolated complex of at least two polypeptides encoded by two polynucleotides wherein said two polypeptides are associated in the complex by affinity binding and are depicted in SEQ ID Nos. 2 and 4.

In yet another embodiment, the present invention relates to an isolated complex comprising at least a polypeptide of SEQ ID Nos. 2 and 4 and variants thereof and a polynucleotides encoding the polypeptides of SEQ ID Nos. 2 and 4 and variants thereof. The present invention is not limited to these polypeptide complexes alone but also includes the isolated complex of the two polypeptides in which fragments and/or homologous polypeptides exhibiting at least 95% sequence identity, as well as from 96% sequence identity to 99.999% sequence identity.

Also encompassed in another embodiment of the present invention is an isolated complex in which the SID® of the prey polypeptides forming the isolated complex.

Besides the isolated complexes described above, nucleic acids coding for a Selected Interacting Domain (SID®) polypeptide or a variant thereof that can be inserted into an expression vector which contains the necessary elements for the transcription and translation of the inserted protein-coding sequence. Such transcription elements include a regulatory region and a promoter. Thus, the nucleic acid which may encode a marker compound of the present invention is operably linked to a promoter in the expression vector. The expression vector may also include a replication origin.

A wide variety of host/expression vector combinations are employed in expressing the nucleic acids of the present invention. Useful expression vectors that can be used include, for example, segments of chromosomal, non-chromosomal and synthetic DNA sequences. Suitable vectors include, but are not limited to, derivatives of SV40 and pcDNA and known bacterial plasmids such as col El, pCR1, pBR322, pMal-C2, pET, pGEX as described by Smith et al (1988), pMB9 and derivatives thereof, plasmids such as RP4, phage DNAs such as the numerous derivatives of phage I such as NM989, as well as other phage DNA such as Ml 3 and filamentous single stranded phage DNA; yeast plasmids such as the 2 micron plasmid or derivatives of the 2m plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like.

For example in a baculovirus expression system, both non-fusion transfer vectors, such as, but not limited to pVL941 (BamHI cloning site Summers [need cite], pVL1393 (BamHI, SmaI, XbaI, EcoRI, NotI, XmaIII, BgIII and PstI cloning sites; Invitrogen) pVL1392 (BgIII, PstI, NotI, XmaIII, EcoRI, XbaII, SmaI and BamHI cloning site; Summers and Invitrogen) and pBlueBacIII (BamHI, BgIII, PstI, NcoI and HindIII cloning site, with blue/white recombinant screening, Invitrogen), and fusion transfer vectors such as, but not limited to, pAc700 (BamHI and KpnI cloning sites, in which the BamHI recognition site begins with the initiation codon; Summers), pAc701 and pAc70-2 (same as pAc700, with different reading frames), pAc360 (BamHI cloning site 36 base pairs downstream of a polyhedrin initiation codon; Invitrogen (195)) and pBlueBacHisA, B, C (three different reading frames with BamHI, BgIII, PstI, NcoI and HindIII cloning site, an N-terminal peptide for ProBond purification and blue/white recombinant screening of plaques; Invitrogen (220) can be used.

Mammalian expression vectors contemplated for use in the invention include vectors with inducible promoters, such as the dihydrofolate reductase promoters, any expression vector with a DHFR expression cassette or a DHFR/methotrexate co-amplification vector such as pED (PstI, SalI, SbaI, SmaI and EcoRI cloning sites, with the vector expressing both the cloned gene and DHFR; Kaufman, 1991). Alternatively a glutamine synthetase/methionine sulfoximine co-amplification vector, such as pEE14 (HindIII, XbalI, SmaI, SbaI, EcoRI and BclI cloning sites in which the vector expresses glutamine synthetase and the cloned gene; Celltech). A vector that directs episomal expression under the control of the Epstein Barr Virus (EBV) or nuclear antigen (EBNA) can be used such as pREP4 (BamHI, SfiI, XhoI, NotI, NheI, HindIII, NheI, PvuII and KpnI cloning sites, constitutive RSV-LTR promoter, hygromycin selectable marker; Invitrogen) pCEP4 (BamHI, SfiI, XhoI, NotI, NheI, HindIII, NheI, PvuII and KpnI cloning sites, constitutive hCMV immediate early gene promoter, hygromycin selectable marker; Invitrogen), pMEP4 (KpnI, PvuI, NheI, HindIII, NotI, XhoI, SfiI, BamHI cloning sites, inducible methallothionein IIa gene promoter, hygromycin selectable marker, Invitrogen), pREP8 (BamHI, XhoI, NotI, HindIII, NheI and KpnI cloning sites, RSV-LTR promoter, histidinol selectable marker; Invitrogen), pREP9 (KpnI, NheI, HindIII, NotI, XhoI, SfiI, BamHI cloning sites, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen).

Selectable mammalian expression vectors for use in the invention include, but are not limited to, pRc/CMV (HindIII, BstXI, NotI, SbaI and ApaI cloning sites, G418 selection, Invitrogen), pRc/RSV (HindIII, SpeI, BstXI, NotI, XbaI cloning sites, G418 selection, Invitrogen) and the like. Vaccinia virus mammalian expression vectors (see, for example Kaufman 1991 that can be used in the present invention include, but are not limited to, pSC11 (SmaI cloning site, TK- and β-gal selection), pMJ601 (SalI, SmaI, AfiI, NarI, BspMII, BamHI, ApaI, NheI, SacII, KpnI and HindIII cloning sites; TK- and β-gal selection), pTKgptF1S (EcoRI, PstI, SalII, AccI, HindIII, SbaI, BamHI and Hpa cloning sites, TK or XPRT selection) and the like.

Yeast expression systems that can also be used in the present include, but are not limited to, the non-fusion pYES2 vector (XbaI, SphI, ShoI, NotI, GstXI, EcoRI, BstXI, BamHI, SacI, KpnI and HindIII cloning sites, Invitrogen), the fusion pYESHisA, B, C (XbalI, SphI, ShoI, NotI, BstXI, EcoRI, BamHI, SacI, KpnI and HindIII cloning sites, N-terminal peptide purified with ProBond resin and cleaved with enterokinase; Invitrogen), pRS vectors and the like.

Consequently, mammalian and typically human cells, as well as bacterial, yeast, fungi, insect, nematode and plant cells an used in the present invention and may be transfected by the nucleic acid or recombinant vector as defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli, (e.g., strain DH5-c), Bacillus subtilis, Salmonella typhimurium, or strains of the genera of Pseudomonas, Streptomyces and Staphylococcus.

Further suitable cells that can be used in the present invention include yeast cells such as those of Saccharomyces such as Saccharomyces cerevisiae.

Besides the specific isolated complexes, as described above, the present invention relates to and also encompasses SID® polynucleotides. As explained above, for each bait polypeptide, several prey polypeptides may be identified by comparing and selecting the intersection of every isolated fragment that are included in the same polypeptide, as described, for example, by Szabo et al, supra.

The present invention is not limited to the SID® sequences as described in the above paragraph, but also includes fragments of these sequences having at least 12 consecutive nucleic acids, between 12 and 5,000 consecutive nucleic acids and between 12 and 10,000 consecutive nucleic acids and between 12 and 20,000 consecutive nucleic acids, as well as variants thereof. The fragments or variants of the SID® sequences possess at least the same affinity of binding to its protein or polypeptide counterpart, against which it has been initially selected. Moreover this variant and/or fragments of the SID® sequences alternatively can have between 95% and 99.999% sequence identity to its protein or polypeptide counterpart.

According to the present invention the variants can be created by known mutagenesis techniques either in vitro or in vivo. Such a variant can be created such that it has altered binding characteristics with respect to the target protein and more specifically that the variant binds the target sequence with either higher or lower affinity.

Polynucleotides that are complementary to the above sequences which include the polynucleotides of the SID®'s, their fragments, variants and those that have specific sequence identity are also included in the present invention.

The polynucleotide encoding the SID® polypeptide, fragment or variant thereof can also be inserted into recombinant vectors which are described in detail above.

The present invention also relates to a composition comprising the above-mentioned recombinant vectors containing the SID® polypeptides fragments or variants thereof, as well as recombinant host cells transformed by the vectors. The recombinant host cells that can be used in the present invention were discussed in greater detail above.

The compositions comprising the recombinant vectors can contain physiological acceptable carriers such as diluents, adjuvants, excipients and any vehicle in which this composition can be delivered therapeutically and can include, but is are not limited to sterile liquids such as water and oils.

In yet another embodiment, the present invention relates to a method of selecting modulating compounds, as well as the modulating molecules or compounds themselves which may be used in a pharmaceutical composition. These modulating compounds may act as a cofactor, as an inhibitor, as antibodies, as tags, as a competitive inhibitor, as an activator or alternatively have agonistic or antagonistic activity on the protein-protein interactions.

The activity of the modulating compound does not necessarily, for example, have to be 100% activation or inhibition. Indeed, even partial activation or inhibition can be achieved that is of pharmaceutical interest.

The modulating compound can be selected according to a method which comprises:

(a) cultivating a recombinant host cell with a modulating compound on a selective medium and a reporter gene the expression of which is toxic for said recombinant host cell wherein said recombinant host cell is transformed with two vectors:

(i) wherein said first vector comprises a polynucleotide encoding a first hybrid polypeptide having a DNA binding domain;

(ii) wherein said second vector comprises a polynucleotide encoding a second hybrid polypeptide having a transcriptional activating domain that activates said toxic reporter gene when the first and second hybrid polypeptides interact;

(b) selecting said modulating compound which inhibits or permits the growth of said recombinant host cell.

In yet another embodiment, the present invention relates to a method of selecting a modulating compound, which modulating compound inhibits the interactions of two polypeptides of βTrCP and RasSF1 and variants of RasSF1. This method comprises:

(a) cultivating a recombinant host cell with a modulating compound on a selective medium and a reporter gene the expression of which is toxic for said recombinant host cell wherein said recombinant host cell is transformed with two vectors:

(i) wherein said first vector comprises a polynucleotide encoding a first hybrid polypeptide having a first domain of an enzyme;

(ii) wherein said second vector comprises a polynucleotide encoding a second hybrid polypeptide having an enzymatic transcriptional activating domain that activates said toxic reporter gene when the first and second hybrid polypeptides interact;

(b) selecting said modulating compound which inhibits or permits the growth of said recombinant host cell.

In the two methods described above any toxic reporter gene can be utilized including those reporter genes that can be used for negative selection including the URA3 gene, the CYH1 gene, the CYH2 gene and the like.

In yet another embodiment, the present invention provides a kit for screening a modulating compound. This kit comprises a recombinant host cell which comprises a reporter gene the expression of which is toxic for the recombinant host cell. The host cell is transformed with two vectors. The first vector comprises a polynucleotide encoding a first hybrid polypeptide having a DNA binding domain; and a second vector comprises a polynucleotide encoding a second hybrid polypeptide having a transcriptional activating domain that activates said toxic reporter gene when the first and second hybrid polypeptides interact.

In yet another embodiment a kit is provided for screening a modulating compound by providing a recombinant host cell, as described in the paragraph above, but instead of a DNA binding domain, the first vector comprises a first hybrid polypeptide containing a first domain of a protein. The second vector comprises a second polypeptide containing a second part of a complementary domain of a protein that activates the toxic reporter gene when the first and second hybrid polypeptides interact.

In the selection methods described above, the activating domain can be p42 Gal 4, YP16 (HSV) and the DNA-binding domain can be derived from Gal4 or Lex A. The protein or enzyme can be adenylate cyclase, guanylate cyclase, DHFR and the like.

In yet another embodiment, the present invention relates to a pharmaceutical composition comprising the modulating compounds for preventing or treating tumors in a human or animal, most preferably in a mammal.

This pharmaceutical composition comprises a pharmaceutically acceptable amount of the modulating compound. The pharmaceutically acceptable amount can be estimated from cell culture assays. For example, a dose can be formulated in animal models to achieve a circulating concentration range that includes or encompasses a concentration point or range having the desired effect in an in vitro system. This information can thus be used to accurately determine the doses in other mammals, including humans and animals.

The therapeutically effective dose refers to that amount of the compound that results in amelioration of symptoms in a patient. Toxicity and therapeutic efficacy of such compounds can be determined by standard pharmaceutical procedures in cell cultures or in experimental animals. For example, the LD50 (the dose lethal to 50% of the population) as well as the ED50 (the dose therapeutically effective in 50% of the population) can be determined using methods known in the art. The dose ratio between toxic and therapeutic effects is the therapeutic index which can be expressed as the ratio between LD 50 and ED50 compounds that exhibit high therapeutic indexes.

The data obtained from the cell culture and animal studies can be used in formulating a range of dosage of such compounds which lies preferably within a range of circulating concentrations that include the ED50 with little or no toxicity.

The pharmaceutical composition can be administered via any route such as locally, orally, systemically, intravenously, intramuscularly, mucosally, using a patch and can be encapsulated in liposomes, microparticles, microcapsules, and the like. The pharmaceutical composition can be embedded in liposomes or even encapsulated.

Any pharmaceutically acceptable carrier or adjuvant can be used in the pharmaceutical composition. The modulating compound will be preferably in a soluble form combined with a pharmaceutically acceptable carrier. The techniques for formulating and administering these compounds can be found in “Remington's Pharmaceutical Sciences” Mack Publication Co., Easton, Pa., latest edition.

The mode of administration optimum dosages and galenic forms can be determined by the criteria known in the art taken into account the seriousness of the general condition of the mammal, the tolerance of the treatment and the side effects.

In yet another embodiment, the present invention relates to a pharmaceutical composition comprising a SID® polypeptide, a fragment or variant thereof. The SID® polypeptide, fragment or variant thereof can be used in a pharmaceutical composition provided that it is endowed with highly specific binding properties to a bait polypeptide of interest.

The original properties of the SID® polypeptide or variants thereof interfere with the naturally occurring interaction between a first protein and a second protein within the cells of the organism. Thus, the SID® polypeptide binds specifically to either the first polypeptide or the second polypeptide.

Thus, the present invention relates to a pharmaceutical composition comprising a pharmaceutically acceptable amount of a SID® polypeptide or variant thereof, provided that the variant has the above-mentioned two characteristics; i.e., that it is endowed with highly specific binding properties to a bait polypeptide of interest and is devoid of biological activity of the naturally occurring protein.

In yet another embodiment, the present invention relates to a pharmaceutical composition comprising a pharmaceutically effective amount of a polynucleotide encoding a SID® polypeptide or a variant thereof wherein the polynucleotide is placed under the control of an appropriate regulatory sequence. Appropriate regulatory sequences that are used are polynucleotide sequences derived from promoter elements and the like.

Polynucleotides that can be used in the pharmaceutical composition of the present invention include the nucleotide sequences of SEQ ID Nos.1 and 3.

Besides the SID® polypeptides and polynucleotides, the pharmaceutical composition of the present invention can also include a recombinant expression vector comprising the polynucleotide encoding the SID® polypeptide, fragment or variant thereof.

The above described pharmaceutical compositions can be administered by any route such as orally, systemically, intravenously, intramuscularly, intradermally, mucosally, encapsulated, using a patch and the like. Any pharmaceutically acceptable carrier or adjuvant can be used in this pharmaceutical composition.

The SID® polypeptides as active ingredients will be preferably in a soluble form combined with a pharmaceutically acceptable carrier. The techniques for formulating and administering these compounds can be found in “Remington's Pharmaceutical Sciences” supra.

The amount of pharmaceutically acceptable SID® polypeptides can be determined as described above for the modulating compounds using cell culture and animal models.

Such compounds can be used in a pharmaceutical composition to treat or prevent tumors.

In another embodiment the present invention nucleic acids comprising a sequence which encodes the RasSF1 protein and/or variants thereof are administered to modulate the βTrCP and RasSF1 complex function by way of gene therapy. Any of the methodologies relating to gene therapy available within the art may be used in the practice of the present invention such as those described by Goldspiel et al Clin. Pharm. 12 pgs. 488-505 (1993).

Delivery of the therapeutic nucleic acid into a patient may be direct in vivo gene therapy (i.e., the patient is directly exposed to the nucleic acid or nucleic acid-containing vector) or indirect ex vivo gene therapy (i.e., cells are first transformed with the nucleic acid in vitro and then transplanted into the patient).

For example for in vivo gene therapy, an expression vector containing the nucleic acid is administered in such a manner that it becomes intracellular; i.e., by infection using a defective or attenuated retroviral or other viral vectors as described, for example in U.S. Pat. No. 4,980,286 or by Robbins et al, Pharmacol. Ther., 80 No. 1 pgs. 35-47 (1998).

The various retroviral vectors that are known in the art are such as those described in Miller et al, Meth. Enzymol. 217 pgs. 581-599 (1993) which have been modified to delete those retroviral sequences which are not required for packaging of the viral genome and subsequent integration into host cell DNA. Also adenoviral vectors can be used which are advantageous due to their ability to infect non-dividing cells and such high-capacity adenoviral vectors are described in Kochanek, Human Gene Therapy, 10, pgs. 2451-2459 (1999). Chimeric viral vectors that can be used are those described by Reynolds et al, Molecular Medicine Today, pgs. 25-31 (1999). Hybrid vectors can also be used and are described by Jacoby et al, Gene Therapy, 4, pgs. 1282-1283 (1997).

Direct injection of naked DNA or through the use of microparticle bombardment (e.g., Gene Gun®; Biolistic, Dupont). or by coating it with lipids can also be used in gene therapy. Cell-surface receptors/transfecting agents or through encapsulation in liposomes, microparticles or microcapsules or by administering the nucleic acid in linkage to a peptide which is known to enter the nucleus or by administering it in linkage to a ligand predisposed to receptor-mediated endocytosis (See, Wu & Wu, J. Biol. Chem., 262 pgs. 4429-4432 (1987)) can be used to target cell types which specifically express the receptors of interest.

In another embodiment a nucleic acid ligand compound may be produced in which the ligand comprises a fusogenic viral peptide designed so as to disrupt endosomes, thus allowing the nucleic acid to avoid subsequent lysosomal degradation. The nucleic acid may be targeted in vivo for cell specific endocytosis and expression by targeting a specific receptor such as that described in WO92/06180, WO93/14188 and WO 93/20221. Alternatively the nucleic acid may be introduced intracellularly and incorporated within the host cell genome for expression by homologous recombination. See, Zijistra et al, Nature, 342, pgs. 435-428 (1989).

In ex vivo gene a gene is transferred into cells in vitro using tissue culture and the cells are delivered to the patient by various methods such as injecting subcutaneously, application of the cells into a skin graft and the intravenous injection of recombinant blood cells such as hematopoietic stem or progenitor cells.

Cells into which a nucleic acid can be introduced for the purposes of gene therapy include, for example, epithelial cells, endothelial cells, keratinocytes, fibroblasts, muscle cells, hepatocytes and blood cells. The blood cells that can be used include, for example, T-lymphocytes, B-lymphocytes, monocytes, macrophages, neutrophils, eosinophils, megakaryotcytes, granulocytes, hematopoietic cells or progenitor cells and the like.

In yet another embodiment the present invention relates to protein chips or protein microarrays. It is well known in the art that microarrays can contain more than 10,000 spots of a protein that can be robotically deposited on a surface of a glass slide or nylon filter. The proteins attach covalently to the slide surface, yet retain their ability to interact with other proteins or small molecules in solution. In some instances the protein samples can be made to adhere to glass slides by coating the slides with an aldehyde-containing reagent that attaches to primary amines. A process for creating microarrays is described, for example by MacBeath and Schreiber in Science, Volume 289, Number 5485, pgs, 1760-1763 (2000) or Service, Science, Vol, 289, Number 5485 pg. 1673 (2000). An apparatus for controlling, dispensing and measuring small quantities of fluid is described, for example, in U.S. Pat. No. 6,112,605.

The present invention also provides a record of protein-protein interactions and any data encompassed in the following Tables. It will be appreciated that this record can be provided in paper or electronic or digital form.

The present invention also relates to the identification of specific domains required on RasSF1 for interaction with β-TrCP. It was discovered that this specific domain is a domain which is common between RasSF1A and RasSF1C. This specific domain was identified via the two hybrid system and co-immunoprecipitation experiments. Also identified were the amino acids not involved in the interaction of RsaSF1A and RasSF1C with β-TrCP; i.e., amino acids 1 to 119 of RasSF1A and amino acids 1 to 49 of RasSF1C, which corresponds to sequences not identical between RasSF1A and RasSF1C.

In yet another aspect of the present invention β-TrCP sequences were also identified that did not interact with RasSF1C. Sequences of β-TrCP having amino acids 1 to 143 (β-TrCPΔN) or amino acids 32 to 179 (β-TrCP-AF) deleted via mutagenesis were tested to determine whether they interact with RasSF1. It was demonstrated that the N-terminal fragment of β-TrCP between amino acids 1 to 179 is not needed for interaction with RasSf1.

In yet another embodiment a more specific domain was identified on β-TrCP which is required for interaction with RasSF1C. This domain is the first WD repeat between amino acids 260 to 291. However, this sequence has to be fused to the N-terminal protein of β-TrCP which precedes the seven WD repeats located at the C-terminus in order to confer the ability to associate with RasSF1.

In yet another embodiment of the present invention RasSF1 acts as a negative modulator of the activity of β-TrCP in the control of expression level and stability of an important substrate of β-TrCP, which is β-catenin (Hart et al 1993). This interaction of RasSF1 with β-TrCP influences the activity of RasSF1 and its tumor suppressive functions in lung, breast and ovarian tumors. Hence, the precise mapping of the interaction domains on both proteins can be used to modulate the function of RasSF1 in tumorigenesis in breast, lung and ovarian tumors in which inactivation of RasSF1 has been associated with the cancer process (Dammann et al 2000, Agathanggelou et al 2000).

In order to fully illustrate the present invention and advantages thereof, the following specific examples are given, it being understood that the same are intended only as illustrative and in nowise limitative.

### EXAMPLES

### Example 1

### Preparation of a Collection of Random-Primed cDNA Fragments

1.A. Collection Preparation and Transformation in Escherichia coli

1.A.1. Random-Primed cDNA Fragment Preparation

For each mRNA sample (human placenta, undifferentiated PAZ6 adipocytes or differentiated PAZ6 adipocytes), random-primed cDNA was prepared from 5 μg of polyA+mRNA using a TimeSaver cDNA Synthesis Kit (Amersham Pharmacia Biotech) and with 5 μg of random N9-mers according to the manufacturer's instructions. Following phenolic extraction, the cDNA was precipitated and resuspended in water. The resuspended cDNA was phosphorylated by incubating in the presence of T4 DNA Kinase (Biolabs) and ATP for 30 minutes at 37° C. The resulting phosphorylated cDNA was then purified over a separation column (Chromaspin TE 400, Clontech), according to the manufacturer's protocol.

1.A.2. Ligation of Linkers to Blunt-Ended cDNA

Oligonucleotide HGX931 (5′ end phosphorylated) 1 μg/μl and HGX932 1 μg/μl.

Sequence of the oligo HGX931: 5′-GGGCCACGM-3′ (SEQ ID No. 5)

Sequence of the oligo HGX932: 5′-TTCGTGGCCCCTG-3′ (SEQ ID No. 6)

Linkers were preincubated (5 minutes at 95° C., 10 minutes at 68° C., 15 minutes at 42° C.) then cooled down at room temperature and ligated with cDNA fragments at 16° C. overnight.

Linkers were removed on a separation column (Chromaspin TE 400, Clontech), according to the manufacturer's protocol.

### 1.A. 3. Vector Preparation

Plasmid pP6 (see FIG. 10) was prepared by replacing the SpellXhoI fragment of pGAD3S2X with the double-stranded oligonucleotide:

| | |
|---|---|
| 5′CTAGCCATGGCCGCAGGGGCCGCGGCCGCACTAGTGGGGATCCTTAATTAA | (SEQ ID No.7) |
| AGGGCCACTGGGGCCCCC | |
| GGTACCGGCGTCCCCGGCGCCGGCGTGATCACCCCTAGGAATTAATTTCCCG | |
| GTGACCCCGGGGGAGCT 3′ | |

The pP6 vector was successively digested with Sfi1 and BamHI restriction enzymes (Biolabs) for 1 hour at 37° C., extracted, precipitated and resuspended in water. Digested plasmid vector backbones were purified on a separation column (Chromaspin TE 400, Clontech), according to the manufacturer's protocol.

1.A.4. Ligation Between Vector and Insert of cDNA

The prepared vector was ligated overnight at 15° C. with the blunt-ended cDNA described in section 2 using T4 DNA ligase (Biolabs). The DNA was then precipitated and resuspended in water.

1.A.5. Library Transformation in Escherichia coli

The DNA from section 1.A.4 was transformed into Electromax DHIOB electrocompetent cells (Gibco BRL) with a Cell Porator apparatus (Gibco BRL). 1 ml SOC medium was added and the transformed cells were incubated at 37° C. for 1 hour. 9 mis of SOC medium per tube was added and the cells were plated on LB+ampicillin medium. The colonies were scraped with liquid LB medium, aliquoted and frozen at −80° C.

The obtained collections of recombinant cell clones were named: HGXBPLARP1 (placenta), HGXBPZURP1 (undifferentiated PAZ6 adipocytes) and HGXBPZDRP1 (differentiated PAZ6 adipocytes).

1.B. Collection transformation in Saccharomyces cerevisiae

The Saccharomyces cerevisiae strain (Y187 (MATα Gal4Δ Gal80Δ ade2-101, his3, leu2-3, -112, trp1-901, ura3-52 URA3::UASGAL1-LacZ Met)) was transformed with the cDNA library.

The plasmid DNA contained in E. coli were extracted (Qiagen) from aliquoted E. coli frozen cells (1.A.5.). Saccharomyces cerevisiae yeast Y187 in YPGlu were grown.

Yeast transformation was performed according to standard protocol (Giest et al. Yeast, 11, 355-360, 1995) using yeast carrier DNA (Clontech). This experiment leads to 10⁴ to 5×10⁴ cells/μg DNA. 2×10⁴ cells were spread on DO-Leu medium per plate. The cells were aliquoted into vials containing 1 ml of cells and frozen at −80° C.

The obtained collections of recombinant cell clones are named: HGXYPLARP1 (placenta), HGXYPZURPI (undifferentiated PAZ6 adipocytes) and HGXYPZDRP1 (differentiated PAZ6 adipocytes).

### 1.C. Construction of Bait Plasmids

For fusions of the bait protein to the DNA-binding domain of the GAL4 protein of S. cerevisiae, bait fragments were cloned into plasmid pB6. For fusions of the bait protein to the DNA-binding domain of the LexA protein of E. coli, bait fragments were cloned into plasmid pB20.

Plasmid pB6 (see FIG. 3) was prepared by replacing the Nco1/Sal1 polylinker fragment of PASΔΔ with the double-stranded DNA fragment:

| | |
|---|---|
| 5′CATGGCCGGACGGGCCGCGGCCGCACTAGTGGGGATCCTTAATTAAAGGGCC | (SEQ ID No.8) |
| ACTGGGGCCCCC 3′ | |
| 3′CGGCCTGCCCGGCGCCGGCGTGATCACCCCTAGGAATTAATTTCCCGGTGACC | |
| CCGGGGGAGCT 5′ | |

Plasmid pB20 (see FIG. 6) was prepared by replacing the EcoRIPstI polylinker fragment of pLex10 with the double-stranded DNA fragment:

| | |
|---|---|
| 5′AATTCGGGGCCGGACGGGCCGCGGCCGCACTAGTGGGGATCCTTAATTAAGG | (SEQ ID No.9) |
| GCCACTGGGGCCCCTCGACCTGCA 3′ | |
| 3′GCCCCGGCCTGCCCGGCGCCGGCGTGATCACCCCTAGGAATTAATTCCCGGT | |
| GACCCCGGGGAGCTGG 5′ | |

### 1.C. Construction of Bait Plasmid

The amplification of the bait ORF was obtained by PCR using the Pfu proof-reading Taq polymerase (Stratagene), 10 pmol of each specific amplification primer and 200 ng of plasmid DNA as template.

The PCR program was set up as follows:

| | | |
|---|---|---|
| 94° | 45″ | |
| 94° | 45″ | |
| 48° | 45″ | {close oversize bracket} × 30 cycles |
| 72° | 6′ | |
| 72° | 10′ | |
| 15° | ∞ | |

The amplification was checked by agarose gel electrophoresis.

The PCR fragments were purified with Qiaquick column (Qiagen) according to the manufacturer's protocol.

Purified PCR fragments were digested with adequate restriction enzymes. The PCR fragments were purified with Qiaquick column (Qiagen) according to the manufacturer's protocol.

The digested PCR fragments were ligated into an adequately digested and dephosphorylated bait vector (pB6 or pB20) according to standard protocol (Sambrook et al.) and were transformed into competent bacterial cells. The cells were grown, the DNA extracted and the plasmid was sequenced.

### Example 2

### Screening the Collection with the Two-Hybrid in Yeast System

2.A. The Mating Protocol

The mating two-hybrid in yeast system (as described by Legrain et al., Nature Genetics, vol. 16, 277-282 (1997), Toward a functional analysis of the yeast genome through exhaustive two-hybrid screens) was used for its advantages but one could also screen the cDNA collection in classical two-hybrid system as described in Fields et al. or in a yeast reverse two-hybrid system.

The mating procedure allows a direct selection on selective plates because the two fusion proteins are already produced in the parental cells. No replica plating is required.

This protocol was written for the use of the library transformed into the Y187 strain.

For bait proteins fused to the DNA-binding domain of GAL4, bait-encoding plasmids were first transformed into S. cerevisiae (CG1945 strain (MATa Gal4-542 Gal180-538 ade2-101 his3Δ200, leu2-3, 112, trp1-901, ura3-52, lys2-801, URA3::GAL4 17mers (X3)-CyC1TATA-LacZ, LYS2::GAL1UAS-GAL1TATA-HIS3CYH^{R})) according to step 1.B. and spread on DO-Trp medium.

For bait proteins fused to the DNA-binding domain of LexA, bait-encoding plasmids were first transformed into S. cerevisiae (L40Δgal4 strain (MATa ade2, trp1-901, leu2 3,112, lys2-801, his3A200, LYS2::(lexAop)₄-HIS3, ura3-52::URA3 (lexAop)₈-LacZ, GAL4::Kan^{R})) according to step 1.B. and spread on DO-Trp medium. Day 1, morning:preculture

The cells carrying the bait plasmid obtained at step 1.C. were precultured in 20 ml DO-Trp medium and grown at 30° C. with vigorous agitation. Day 1, late afternoon: culture

The OD₆₀₀ₙₘ of the DO-Trp pre-culture of cells carrying the bait plasmid pre-culture was measured. The OD₆₀₀ₙₘ must lie between 0.1 and 0.5 in order to correspond to a linear measurement.

50 ml DO-Trp at OD₆₀₀ₙₘ 0.006/ml was inoculated and grown overnight at 30° C. with vigorous agitation.

Day 2: mating

medium and plates

1 YPGlu 15 cm plate

50 ml tube with 13 ml DO-Leu-Trp-His

100 ml flask with 5 ml of YPGlu

8 DO-Leu-Trp-His plates

2 DO-Leu plates

2 DO-Trp plates

2 DO-Leu-Trp plates

The OD₆₀₀ₙₘ of the DO-Trp culture was measured. It should be around 1.

For the mating, twice as many bait cells as library cells were used. To get a good mating efficiency, one must collect the cells at 10⁸ cells per cm².

The amount of bait culture (in ml) that makes up 50 OD₆₀₀ₙₘ units for the mating with the prey library was estimated.

A vial containing the HGXYcDNA1 library was thawed slowly on ice. 1.0 ml of the vial was added to 5 ml YPGlu. Those cells were recovered at 30° C., under gentle agitation for 10 minutes.

Mating

The 50 OD₆₀₀ₙₘ units of bait culture was placed into a 50 ml falcon tube.

The HGXYcDNA1 library culture was added to the bait culture, then centrifuged, the supernatant discarded and resuspended in 1.6 ml YPGlu medium. The cells were distributed onto two 15 cm YPGlu plates with glass beads. The cells were spread by shaking the plates. The plate cells-up at 30° C. for 4 h30 min were incubated.

Collection of Mated Cells

The plates were washed and rinsed with 6 ml and 7 ml respectively of DO-Leu-Trp-His. Two parallel serial ten-fold dilutions were performed in 500 μl DO-Leu-Trp-His up to 1/10,000. 50 μl of each 1/10000 dilution was spread onto DO-Leu and DO-trp plates and 50 μl of each 1/1000 dilution onto DO-Leu-Trp plates. 22.4 ml of collected cells were spread in 400 μl aliquots on DO-Leu-Trp-His+Tet plates.

Day 4

Clones that were able to grow on DO-Leu-Trp-His+Tetracyclin were then selected. This medium allows one to isolate diploid clones presenting an interaction.

The His+colonies were counted on control plates.

The number of His+cell clones will define which protocol is to be processed:

Upon 60.10⁶ Trp+Leu+colonies:

if the number His+cell clones <285: then use the process luminometry protocol on all colonies

if the number of His+cell clones >285 and <5000: then process via overlay and then luminometry protocols on blue colonies (2.B and 2.C).

if number of His+cell clones >5000: repeat screen using DO-Leu-Trp-His+Tetracyclin plates containing 3-aminotriazol.

2.B. The X-Gal Overlay Assay

The X-Gal overlay assay was performed directly on the selective medium plates after scoring the number of His⁺ colonies.

### Materials

A waterbath was set up. The water temperature should be 50° C.

0.5 M Na₂HPO₄ pH 7.5.

1.2% Bacto-agar.

2% X-Gal in DMF.

Overlay mixture: 0.25 M Na₂HPO₄ pH 7.5, 0.5% agar, 0.1% SDS, 7% DMF (LABOSI), 0.04% X-Gal (ICN). For each plate, 10 ml overlay mixture are needed.

DO-Leu-Trp-His plates.

Sterile toothpicks.

Experiment

The temperature of the overlay mix should be between 45° C. and 50° C. The overlay-mix was poured over the plates in portions of 10 ml. When the top layer was settled, they were collected. The plates were incubated overlay-up at 30° C. and the time was noted. Blue colonies were checked for regularly. If no blue colony appeared, overnight incubation was performed. Using a pen the number of positives was marked. The positives colonies were streaked on fresh DO-Leu-Trp-His plates with a sterile toothpick.

2. C. The Luminometry Assay

His+ colonies were grown overnight at 30° C. in microtiter plates containing DO-Leu-Trp-His+Tetracyclin medium with shaking. The day after, the overnight culture was diluted 15 times into a new microtiter plate containing the same medium and was incubated for 5 hours at 30° C. with shaking. The samples were diluted 5 times and read OD₆₀₀ₙₘ. The samples were diluted again to obtain between 10,000 and 75,000 yeast cells/well in 100 μl final volume.

Per well, 76 μl of One Step Yeast Lysis Buffer (Tropix) was added, 20 μl Sapphirell Enhancer (Tropix), 4 μl Galacton Star (Tropix) and incubated 40 minutes at 30° C. The β-Gal read-out (L) was measured using a Luminometer (Trilux, Wallach). The value of (OD₆₀₀ₙₘ×L) was calculated and interacting preys having the highest values were selected.

At this step of the protocol, diploid cell clones presenting interaction were isolated. The next step was now to identify polypeptides involved in the selected interactions.

### Example 3

### Identification of Positive Clones

3.A. PCR on Yeast Colonies

Introduction

PCR amplification of fragments of plasmid DNA directly on yeast colonies is a quick and efficient procedure to identify sequences cloned into this plasmid. It is directly derived from a published protocol (Wang H. et al., Analytical Biochemistry, 237, 145-146, (1996)). However, it is not a standardized protocol and it varies from strain to strain and it is dependent of experimental conditions (number of cells, Taq polymerase source, etc). This protocol should be optimized to specific local conditions.

Materials

For 1 well, PCR mix composition was:

32.5 μl water,

5 μl 10× PCR buffer (Pharmacia),

1 μl dNTP 10 mM,

0.5 μl Taq polymerase (5 u/μl) (Pharmacia),

0.5 μl oligonucleotide ABS1 10 pmole/μl: 5′-GCGTTTGGAATCACTACAGG-3′,(SEQ ID No.10)

0.5 μl oligonucleotide ABS2 10 pmole/μl: 5′-CACGATGCACGTTGAAGTG-3′.(SEQ ID No. 11)

1 N NaOH.

Experiment

The positive colonies were grown overnight at 30° C. on a 96 well cell culture cluster (Costar), containing 150 μl DO-Leu-Trp-His+Tetracyclin with shaking. The culture was resuspended and 100 μl was transferred immediately on a Thermowell 96 (Costar) and centrifuged for 5 minutes at 4,000 rpm at room temperature. The supernatant was removed. 5 μl NaOH was added to each well and shaken for 1 minute.

The Thermowell was placed in the thermocycler (GeneAmp 9700, Perkin Elmer) for 5 minutes at 99.9° C. and then 10 minutes at 4° C. In each well, the PCR mix was added and shaken well.

The PCR program was set up as followed:

| | | |
|---|---|---|
| 94° C. | 3 minutes | |
| 94° C. | 30 seconds | |
| 53° C. | 1 minute 30 seconds | {close oversize bracket} × 35 cycles |
| 72° C. | 3 minutes | |
| 72° C. | 5 minutes | |
| 15° C. | ∞ | |

The quality, the quantity and the length of the PCR fragment was checked on an agarose gel. The length of the cloned fragment was the estimated length of the PCR fragment minus 300 base pairs that corresponded to the amplified flanking plasmid sequences.

3. B. Plasmids Rescue from Yeast by Electroporation

### Introduction

The previous protocol of PCR on yeast cell may not be successful, in such a case, plasmids from yeast by electroporation can be rescued. This experiment allows the recovery of prey plasmids from yeast cells by transformation of E. coli with a yeast cellular extract. The prey plasmid can then be amplified and the cloned fragment can be sequenced.

### Materials

Plasmid rescue

Glass beads 425-600 μm (Sigma)

Phenol/chloroform (1/1) premixed with isoamyl alcohol (Amresco)

Extraction buffer: 2% Triton X100, 1% SDS, 100 mM NaCl, 10 mM TrisHCl pH 8.0, 1 mM EDTA pH 8.0.

Mix ethanol/NH₄Ac:6 volumes ethanol with 7.5 M NH₄ Acetate, 70% Ethanol and yeast cells in patches on plates.

Electroporation

SOC medium

M9 medium

Selective plates: M9-Leu+Ampicillin

2 mm electroporation cuvettes (Eurogentech)

### Experiment

Plasmid rescue

The cell patch on DO-Leu-Trp-His was prepared with the cell culture of section 2.C. The cell of each patch was scraped into an Eppendorf tube, 300 μl of glass beads was added in each tube, then, 200 μl extraction buffer and 200 μl phenol:chloroform:isoamyl alcohol (25:24:1) was added.

The tubes were centrifuged for 10 minutes at 15,000 rpm. 180 μl supernatant was transferred to a sterile Eppendorf tube and 500 μl each of ethanol/NH₄Ac was added and the tubes were vortexed. The tubes were centrifuged for 15 minutes at 15,000 rpm at 4° C. The pellet was washed with 200 μl 70% ethanol and the ethanol was removed and the pellet was dried. The pellet was resuspended in 10 μl water. Extracts were stored at −20° C.

Electroporation

Materials: Electrocompetent MC1066 cells prepared according to standard protocols (Sambrook et al. supra).

1 μl of yeast plasmid DNA-extract was added to a pre-chilled Eppendorf tube, and kept on ice.

1 μl plasmid yeast DNA-extract sample was mixed and 20 μl electrocompetent cells was added and transferred in a cold electroporation cuvette. Set the Biorad electroporator on 200 ohms resistance, 25 μF capacity; 2.5 kV. Place the cuvette in the cuvette holder and electroporate.

1 ml of SOC was added into the cuvette and the cell-mix was transferred into a sterile Eppendorf tube. The cells were recovered for 30 minutes at 37° C., then spun down for 1 minute at 4,000 × g and the supernatant was poured off. About 100 μl medium was kept and used to resuspend the cells and spread them on selective plates (e.g., M9-Leu plates). The plates were then incubated for 36 hours at 37° C.

One colony was grown and the plasmids were extracted. Check for the presence and size of the insert through enzymatic digestion and agarose gel electrophoresis. The insert was then sequenced.

### Example 4

### Protein-Protein Interaction

For each bait, the previous protocol leads to the identification of prey polynucleotide sequences. Using a suitable software program (e.g., Blastwun, available on the Internet site of the University of Washington: http://bioweb.pasteur.fr/seganal/interfaces/blastwu.html) the identity of the mRNA transcript that is encoded by the prey fragment may be determined and whether the fusion protein encoded is in the same open reading frame of translation as the predicted protein or not.

Alternatively, prey nucleotide sequences can be compared with one another and those which share identity over a significant region (60 nt) can be grouped together to form a contiguous sequence (Contig) whose identity can be ascertained in the same manner as for individual prey fragments described above.

### Example 5

### Identification of SID®

By comparing and selecting the intersection of all isolated fragments that are included in the same polypeptide, one can define the Selected Interacting Domain (SID®).

### Example 6

2-hybrid screens

The N-terminal 219 amino acids of the βTrCP protein (which contain the F-box motif and the first of the WD40 repeats) were fused the DNA-binding domain of the GAL4 protein in the context of the pB6 plasmid (see examples) and used as a bait in yeast two-hybrid screens. In total, three libraries of random-primed cDNA fragments were screened: human placenta (16.5 million independent clones); human undifferentiated PAZ6 adipocytes (10 million independent clones); and human differentiated PAZ6 adipocytes (15 million independent clones). The results of these screens are shown in Table 1.

TABLE 1

| | | | | # Independent |
|---|---|---|---|---|
| Library | | # positives sequenced/ | # RasSF1- | rasSF1-encoding |
| screened | “coverage” | # positives obtained | encoding clones | clones |
| HGXYPLARP1 | 4 times | 228/230 | 7 | 3 |
| HGXYPZURP1 | 7 times | 189/190 | 10 | 6 |
| HGXYPZDRP1 | 4 times | 286/293 | 15 | 7 |

Legend to Table 1

Column 1: Library screened: PLA-RP, Random-primed cDNA prepared from mRNA from human placenta; PZU-RP, Random-primed cDNA prepared from mRNA from undifferentiated human PAZ6 adipocytes; PZD-RP, Random-primed cDNA prepared from mRNA from differentiated human PAZ6 adipocytes.

Column 2: Coverage: Defined as the number of diploid yeast screened divided by the number of independent clones present in the cDNA library.

Column 3: Number of prey sequences analysed compared to the number of His+yeast obtained in each screen.

Column 4: Number of RasSF1-encoding clones for each library. Prey sequences were compared to GenEMBL database using the blastn program.

Column 5: Number independent RasSF1-encoding clones. Both 5 prime and 3 prime extremities of prey fragments were sequenced.

In the cases where it was possible to distinguish the isoform of RasSF1 encoded by the prey fragments (when the prey fragment encoded a protein upstream of the shared C-terminal domain −28/32 fragments) the prey fragments were found to encode the RasSF1C isoform (GI “5524228” [GenBank]). In one case (from the screen against the differentiated adipocyte cDNA library) a novel sequence was found fused to the common domain.

Comparison of the results for all three libraries allows to define the SID for the RasSF1 protein interacting with βTrCP as lying between aa51 and aa270 of the RasSF1C protein. This region is identical between the RasSF1A and RasSF1C isoforms.

| | |
|---|---|
| Bait sequence (beta TrCP) | |
| a tggacccggc cgaggcggtg ctgcaagaga aggcactcaa gtttatgaat | (SEQ ID No.1) |
| tcctcagaga gagaagactg taataatggc gaacccccta ggaagataat accagagaag | |
| aattcactta gacagacata caacagctgt gccagactct gcttaaacca agaaacagta | |
| tgtttagcaa gcactgctat gaagactgag aattgtgtgg ccaaaacaaa acttgccaat | |
| ggcacttcca gtatgattgt gcccaagcaa cggaaactct cagcaagcta tgaaaaggaa | |
| aaggaactgt gtgtcaaata ctttgagcag tggtcagagt cagatcaagt ggaatttgtg | |
| gaacatctta tatcccaaat gtgtcattac caacatgggc acataaactc gtatcttaaa | |
| cctatgttgc agagagattt cataactgct ctgccagctc ggggattgga tcatatcgct | |
| gagaacattc tgtcatacct ggatgccaaa tcactatgtg ctgctgaact tgtgtgcaag | |
| gaatggtacc gagtgacctc tgatggcatg ctgtggaaga agcttatcga gagaatggtc | |
| aggacagatt ctctgtggag aggcctggca gaacgaagag gatgggga ca g tat tta ttc | |
| aaa aac | |
| MDPAEAVLQEKALKFMNSSEREDCNNGEPPRKIIPEKNSLRQTY | (SEQ ID No.2) |
| NSCARLCLNQETVCLASTAMKTENCVAKTKLANGTSSMIVPKQRKLSASYEKEKELCV | |
| KYFEQWSESDQVEFVEHLISQMCHYQHGHINSYLKPMLQRDFITALPARGLDHIAENI | |
| LSYLDAKSLCAAELVCKEWYRVTSDGMLWKKLIERMVRTDSLWRGLAERRGWGQ | |
| YLFK N | |
| Prey Sequence (RasSF1) | |
| 1 ccggggcggt ggttggcggc tacggacgcg caggactggg ggacgggcgg gtacggctat | (SEQ ID No.3) |
| 61 gggcgaggcg gaggcgcctt ctttcgaaat gacctggagc agcacgacga gcagtggcta | |
| 121 ctgcagccaa gaggactcgg actcggagct cgagcagtac ttcaccgcgc gaacctcgct | |
| 181 agctcgcagg ccgcgccggg accaggacga gcctgtggag tgggagacac ctgacctttc | |
| 241 tcaagctgag attgagcaga agatcaagga gtacaatgcc cagatcaaca gcaacctctt | |
| 301 catgagcttg aacaaggacg gttcttacac aggcttcatc aaggttcagc tgaagctggt | |
| 361 gcgccctgtc tctgtgccct ccagcaagaa gccaccctcc ttgcaggatg cccggcgggg | |
| 421 cccaggacgg ggcacaagtg tcaggcgccg cacttccttt tacctgccca aggatgctgt | |
| 481 caagcacctg catgtgctgt cacgcacaag ggcacgtgaa gtcattgagg ccctgctgcg | |
| 541 aaagttcttg gtggtggatg acccccgcaa gtttgcactc tttgagcgcg ctgagcgtca | |
| 601 cggccaagtg tacttgcgga agctgttgga tgatgagcag cccctgcggc tgcggctcct | |
| 661 ggcagggccc agtgacaagg ccctgagctt tgtcctgaag gaaaatgact ctggggaggt | |
| 721 gaactgggac gccttcagca tgcctgaact acataacttc ctacgtatcc tgcagcggga | |
| 781 ggaggaggag cacctccgcc agatcctgca gaagtactcc tattgccgcc agaagatcca | |
| 841 agaggccctg cacgcctgcc cccttgggtg acctcttgta cccccaggtg gaaggcagac | |
| 901 agcaggcagc gccaagtgcg tgccgtgtga gtgtgacagg gccagtgggg cctgtggaat | |
| 961 gagtgtgcat ggaggccctc ctgtgctggg ggaatgagcc cagagaacag cgaagtagct | |
| 1021 tgctccctgt gtccacctat gggtgtagcc aggtatggct ctgcacccct ctgccctcat | |
| 1081 tactgggcct tagtgggcca gggctgccct gagaagctgc tccaggcctg cagcaggagt | |
| 1141 ggtgcagaca gaagtctcct caatttttgt ctcagaagtg aaaatcttgg agaccctgca | |
| 1201 aacagaacag ggtcatgttt gcaggggtga cggccctcat ctatgaggaa aggttttgga | |
| 1261 tcttgaatgt ggtctcagga tatccttatc agagctaagg gtgggtgctc agaataaggc | |
| 1321 aggcattgag gaagagtctt ggtttctctc tacagtgcca actcctcaca caccctgagg | |
| 1381 tcagggagtg ctggctcaca gtacagcatg tgccttaatg cttcatatga ggaggatgtc | |
| 1441 cctgggccag ggtctgtgtg aatgtgggca ctggcccagg ttcatacctt atttgctaat | |
| 1501 caaagccagg gtctctccct caggtgtttt ttatgaagtg cgtgaatgta tgtaatgtgt | |
| 1561 ggtggcctca gctgaatgcc tcctgtgggg aaaggggttg gggtgacagt catcatcagg | |
| 1621 cctggggctg agagaattgg ctcaataaag atttcaagat ccaaaaaaaa aaaaaaaaaa | |
| MGEAEAPSFEMTWSSTTSSGYCSQEDSDSELEQYFTARTSLARR | (SEQ ID No.4) |
| PRRDQDEPVEWETPDLSQAEIEQKIKEYNAQINSNLFMSLNKDGSYTGFIKVQLKLVR | |
| PVSVPSSKKPPSLQDARRGPGRGTSVRRRTSFYLPKDAVKHLHVLSRTRAREVIEALL | |
| RKFLVVDDPRKFALFERAERHGQVYLRKLLDDEQPLRLRLLAGPSDKALSFVLKEN | |
| DS GEVNWDAFSMPELHNFLRILQREEEHLRQILQKYSYCRQKIQEALHACPLG | |

### Example 7

### Making of Polyclonal and Monoclonal Antibodies

The protein-protein complex of βTrCP and RasSF1 was injected into mice and polyclonal and monoclonal antibodies were made following the procedure set forth in Sambrook et al supra.

More specifically, mice are immunized with an immunogen comprising βTrCP and RasSF1 complexes conjugated to keyhole limpet hemocyanin using glutaraldehyde or EDC as is well known in the art. The complexes can also be stabilized by crosslinking as described in WO 00/37483. The immunogen is then mixed with an adjuvant. Each mouse receives four injections of 10 ug to 100 ug of immunogen, and after the fourth injection, blood samples are taken from the mice to determine if the serum contains antibodies to the immunogen. Serum titer is determined by ELISA or RIA. Mice with sera indicating the presence of antibody to the immunogen are selected for hybridoma production.

Spleens are removed from immune mice and single-cell suspension is prepared (Harlow et al, Am. J. Epidemiol. 1988 Apr; 127(4) pgs. 857-63) Cell fusions are performed essentially as described by Kohler and Milstein (1979). Briefly, P365.3 myeloma cells (ATTC Rockville, Md) or NS-1 myeloma cells are fused with spleen cells using polyethylene glycol as described by Harlow et al (1989) Cells are plated at a density of 2×10⁵ cells/well in 96-well tissue culture plates. Individual wells are examined for growth and the supernatants of wells with growth are tested for the presence of the complex-specific antibodies by ELISA or RIA using the βTrCP and RasSF1 complex as a target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to βTrCP and/or RasSF1 to determine which are specific for the βTrCP and RasSF1 complex as opposed to those that bind to the individual proteins.

Monoclonal antibodies against each of the complexes in Table 1 are prepared in a similar manner by mixing specified proteins together, immunizing an animal, fusing spleen cells with myeloma cells and isolating clones which produce antibodies specific for he protein complex, but not for individual proteins.

### Example 8

Ras SF1-βTrCP co-immunoprecipitation and effect of Ras F1 dsRNA 185

1. Co-immunoprecipitation experiments

8×10⁶ 293 cells (ATCC CRL-1573) were transiently transfected by electroporation with 15 μg of vectors expressing either each of the RasSF1 constructs or each of the βTrCP constructs or simultaneously cotransfected with RasSF1 and βTrCP constructs as indicated. Cells were harvested 48 h after transfection and lysed in buffer (50 mM Tris HCl pH 7.5, 1 mM EDTA, 150 mM NaCl, 10% glycerol, 1% Triton X100, 1% antiprotease cocktail (Sigma)). For immunoprecipitations, cell lysates were incubated with 5 μg of rat monoclonal anti-HA per ml (clone 3F10, Roche) or 5 μg of mouse monoclonal anti-myc per ml (clone 9E10, SantaCruz) antibodies for 90 min and then incubated with protein G-Agarose beads (Roche) for 1 h. The beads were washed with lysis buffer. Immune complexes were eluted with Laemmli sample buffer (Sigma), separated by 12% SDS PAGE, and revealed by chemiluminescence using rat anti-HA (clone 3F10, Roche) or mouse anti-Myc (clone 9E10, Santa Cruz).

2. Ras SF1A and Ras SF1 C co-immunoprecipitated with β-TrCP in human cell line 293:

15 μg of Myc-RasSF1A or of Myc-RasSF1C, expressing plasmids were co-transfected in 293 cells by electroporation with 15 μg of HA-βTrCP expressing plasmid (pASI B βTrCP previously described in Lassot et al. 2001). 48 hours after transfection, cells were lysed in lysis buffer (50 mM Tris HCl pH 7.5,1 mM EDTA, 150 mM NaCl, 10% glycerol, 1% Triton X100, 1% antiprotease cocktail from Sigma), and immunoprecipitation was performed with anti-HA antibody 3F10 (Roche) and protein G agarose beads (Sigma) according Lassot et al. 2001). Western blots of the immunoprecipitates were performed using anti myc antibody 9E10 (Roche) or anti-HA antibody 3F10 (Roche) conjugated to peroxidase. As a control of expression, Western blots were also performed with anti myc antibody 9E10 (Roche) or anti-HA antibody 3F10 (Roche) conjugated to peroxidase, directly on the cell lysates of transfected cells before immunoprecipitation.

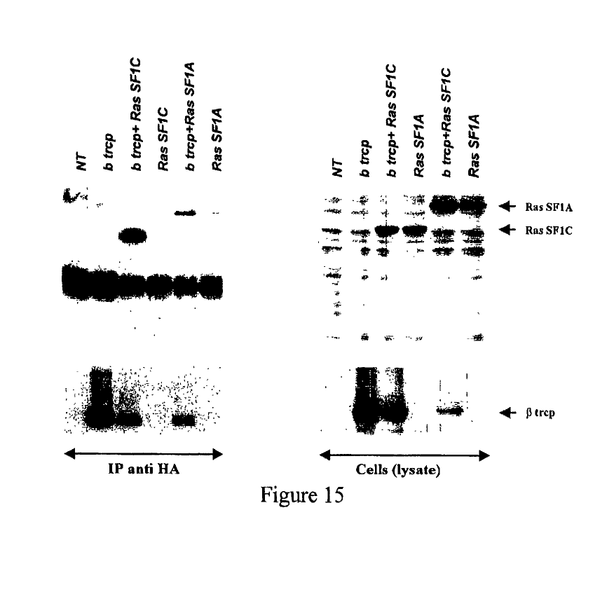

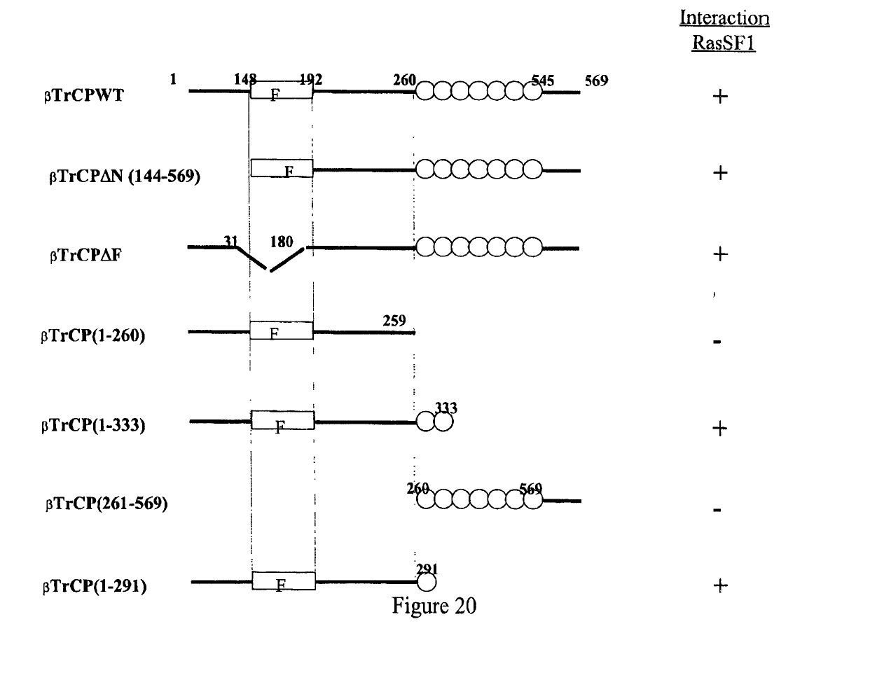

One can see, from FIG. 15 left panel, that by using anti-HA antibodies 3F10, both Myc-Ras SF1 A and Myc-Ras SF1 C are specifically co-immunoprecipitated with HA-βTrCP, since they are not present in the controls in the absence of transfected HA-β-TrCP. On the right panel one can see the results of expression of the different transfected cells before immunoprecipitation, showing that Ras SF1 or β-TrCP are correctly expressed in all samples in which they were transfected. Altogether, these results mean that RasSF1A or RasSF1 C are both capable to associate with βTrCP in human cells. RasSF1 A and RasSF1C share exons 1-5 but differ in their 5′ regions. RasSF1A (accession number AF1 02770) has exons 1A, 1C before exons 1 to 5, while RasSF1C transcription starts at exon 1. Thus RasSF1 A and C share the same protein sequence starting at amino acid 120 of RasSF1A which corresponds to RasSF1C amino acid 50. One can conclude from these two-hybrid results that the domain specifically required on RasSF1 for interaction with β-TrCP is the domain which is in common between RasSF1A and RasSF1C (see FIG. 20). This means that the N-terminal region having 1-119 amino acids from Ras SF1A or 1 to 49 amino acids from RasSF1C is not required for interaction with β-TrCP (Agathanggelou et al. 2000).

### Example 9

1. Plasmid Construction and Mutagenesis.

The RasSF1 A, RasSF1 C and the β-TrCP sequences correspond to the Genbank accession numbers AF102770, AF040703, and Y14153 respectively (Dammann et al. 2000, Agathanggelou et al. 2000, Margottin et al. 1998). Ras SF1A or RasSF1C were subcloned in frame with the C-terminal end of the Myc epitope in the plasmids pMyc-RasSF1A, pMyc-RasSF1C. The βTrCP constructs ( βTrCPΔN: βTrCP 144-569; βTrCP 1-260; βTrCP261-569; βTrCP 1-333) were obtained by PCR on the βTrCP sequence (Margottin F., Bour S., Durand H, Selig, L., Benichou S., Richard V., Thomas D., Strebel K. and Benarous R. A novel human WD protein, h-β-TrCP, that interacts with HIV-1Vpu connects CD4 to the ER degradation pathway through an F-box motif. Mol. Cell, 1, 565-574. 1998), with the following primers:

TrCP(1-260)

| | | |
|---|---|---|
| sense: | GCGAGCGGATCCATGGACCCGGCCGAGGCGGTGCTG | (SEQ ID No.12) |
| antisense: | GCGAGCGTCGACCTAGGGCCCTCTCTGTAAACTATGTCTTCC | (SEQ ID No.13) |

TrCP(261-569)

| | | |
|---|---|---|
| sense: | GCGAGCGGATCCATGATTCACTGCCGAAGTGAAACAAGC | (SEQ ID No.14) |
| Antisense: | GCGAGCGTCGACCTAGGGCCCTTATCTGGAGATGTAGGTGTA | (SEQ ID No.15) |

TrCP(1-333)

| | | |
|---|---|---|
| sense: | GCGAGCGGATCCATGGACCCGGCCGAGGCGGTGCTG | (SEQ ID No.16) |
| antisense: | GCGAGCGTCGACCTAGGGCCCATCCCACACTCTGACCGTGGAATC | (SEQ ID No.17) |

TrCPΔN(144-569)

| | | |
|---|---|---|
| Sense: | GCGAGCGGATCCATGTTCATAACTGCTCTGCCAGCT | (SEQ ID No.18) |
| Antisense: | GCGAGCGTCGACCTAGGGCCCTTATCTGGAGATGTAGGTGTA | (SEQ ID No.19) |

βTrCPΔF was described in Margottin et al. (1998). The PCR fragments were inserted in the pAS1B vector in frame with the C-terminus of the HA epitope (Selig, L., J. C. Pages., V. Tanchou, S. Preveral, C. Berlioz-Torrent, L. X. Liu, L. Erdtmann, J. Darlix, R. Benarous, and S. Benichou. Interaction with the p6 domain of the gag precursor mediates incorporation into virions of Vpr and Vpx proteins from primate lentiviruses J. Virol., 73, 592-600 1999) to generate the corresponding βTrCP constructs fused at their N-terminus with the HA epitope. βTrCP, βTrCPΔN, βTrCPΔF, were subcloned in the pAS1B vector (Selig, L., J. C. Pages., V. Tanchou, S. Preveral, C. Berlioz-Torrent, L. X. Liu, L. Erdtmann, J. Darlix, R. Benarous, and S. Benichou. 1999. J. Virol. 73:592-600): HA-βTrCP, HA-βTrCPΔN 144-569, HA-βTrCPΔF, HA-βTrCP1-260, HA-βTrCP 261-569, HA-βTrCP 1-333.

2. Ras SF1 C can immunoprecipitate with βTrCPΔN or with βTrCPΔF

15 μg of plasmid expressing HA-βTrCP (Lassot et al. 2001), or HA-βTrCPAN (βTrCP144-569), with deletion of amino acids 1-143 of \tilde{β}TrCP made by PCR amplification of HA-βTrCP using appropriate primers to delete amino acids 1-143 of βTrCP), or of HA-β-TrCPΔF (deletion of amino acids 32 to 179 described in Lassot et al. 2001), were co-transfected with 15 μg of plasmid expressing Myc-Ras SF1C, by electroporation of 293 cells as described above in Example 8. 48 hours after transfection, cells were lysed in lysis buffer (50 mM Tris HCl pH 7.5,1 mM EDTA, 150 mM NaCl, 10% glycerol, 1% Triton X100, 1% antiprotease cocktail from Sigma), and immunoprecipitation was performed with anti-HA antibody 3F10 (Roche) and protein G agarose beads (Sigma) according to Lassot et al. 2001). Western blots of the immunoprecipitates were performed using anti myc antibody 9E10 (Roche) or anti-HA antibody 3F10 (Roche) conjugated to peroxidase (right panel). As a control of expression, Western blots were also performed with anti myc antibody 9E10 (Roche) or anti-HA antibody 3F10 (Roche) conjugated to peroxidase, directly on the cell lysates of transfected cells before immunoprecipitation (left panel).

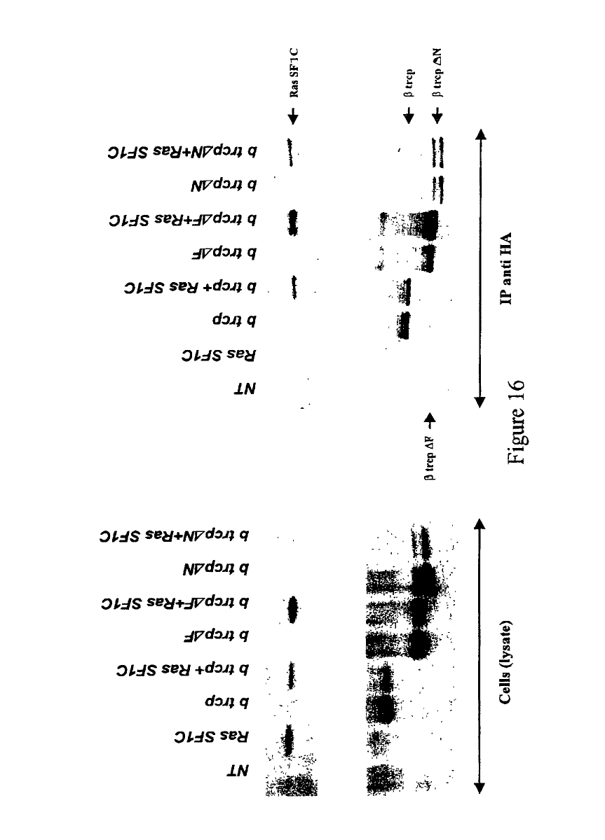

One can see, from FIG. 16 (right panel) that by using anti-HA antibodies 3F10, Myc-RasSF1C is co-immunoprecipitated with HA-βTrCP as well as with HA-βTrCPΔN or with HA-β-TrCPΔF. In the controls without transfection of HA-βTrCP, RasSF1C is not present in the anti-HA immunoprecipitates. These results mean that the sequences which are deleted in \tilde{β}TrCPΔN or in βTrCPΔF (1-143 amino acids or 32-179 amino acids, respectively) are not required for interaction with RasSF1C. Thus one can conclude that the N-terminal fragment of βTrCP between amino acids 1 to 179 is not needed for interaction with RasSF1.

### Example 10

1. Ras SF1 C can Immunoprecipitate with βTrCP 1-333 but not with βTrCP 1-260 or with βTrCP 261-569

Deletion mutants of βTrCP were constructed by PCR amplification using appropriate primers and were subcloned at the 5′BamH1-3′Sal1 restriction sites of the multicloning site in the pAS1 B eucaryotic expression plasmid at the C-terminus and in frame with the HA epitope. These deletion mutants of βTrCP used in this experiment were the following:

βTrCP 1-260 deleted of the C-terminal part of the protein (deletion of amino acid residues 261-569), and thus lacks the seven WD repeats motifs required for the binding of substrates (Vpu, IkBa, β-catenin) to βTrCP;

βTrCP 1-333 composed of the N-terminal part of the βTrCP protein including the first two WD repeats (deletion of amino acid residues 334-569); and

βTrCP 261-569 composed of the seven WD repeats without the N-terminal part of the protein (deletion of amino acid residues 1-260).

15 μg of plasmids expressing HA-βTrCP (Lassot et al. 2001), or HA-βTrCP 1-260, or HA-βTrCP 1-333, or HA-βTrCP 261-569, were co-transfected with 15 μg of plasmid expressing Myc-Ras SF1C, by electroporation of 293 cells as described above in Example 8. 48 hours after transfection, cells were lysed in lysis buffer (50 mM Tris HCl pH 7.5, 1 mM EDTA, 150 mM NaCl, 10% glycerol, 1% Triton X100, 1% antiprotease cocktail from Sigma), and immunoprecipitation was performed with anti-HA antibody 3F10 (Roche) and protein G agarose beads (Sigma) according to Lassot et al. 2001). Western blots of the immunoprecipitates were performed using anti myc antibody 9E10 (Roche) (upper panel) or anti-HA antibody 3F10 (Roche) conjugated to peroxidase (lower panel). As a control of expression, Western blots were also performed with anti myc antibody 9E10 (Roche) conjugated to peroxidase, directly on the cell lysates of transfected cells before immunoprecipitation (middle panel).

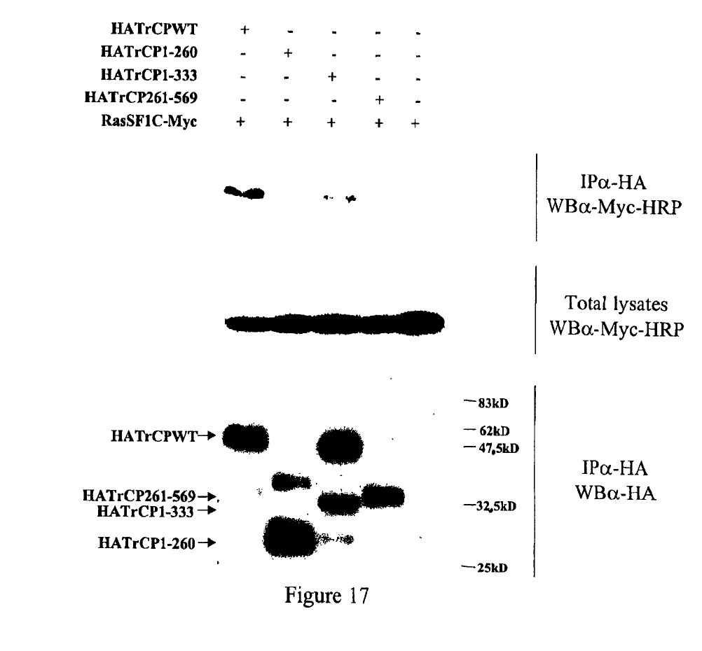

One can see, that by using anti-HA antibodies 3F10, Myc-RasSF1 C is co-immunoprecipitated with HA-βTrCP as well as with HA-βTrCP 1-333, but not with HA-βTrCP 1-260, neither with HA-βTrCP 261-569. The middle panel shows that Ras SF1C is equally expressed in all cells transfected with the RasSF1C expression plasmid. In the control without transfection of HA-βTrCP, RasSF1C is not present in the anti-HA immunoprecipitate. From these co-immunoprecipitation experiments showed in FIG. 17, one can conclude that the region of βTrCP required for interaction with RasSF1C is between amino acids 260 to 333, which encodes the first two WD repeats.

Taken together with the results of two-hybrid studies showing that RasSF1 interacts with the N-terminal part of βTrCP from amino acids 1 to 291, one can conclude that in fact the sequence required on βTrCP for interaction with RasSF1C is the sequence of the first WD repeat between amino acids 260 to 291. Interestingly, this sequence has to be fused to the N-terminal portion of βTrCP which precedes the seven WD repeats located at the C-terminus, in order to confer the ability to associate with RasSF1. As shown in FIG. 17, the sequence 261-569 which encodes all the seven WD repeats of βTrCP but which lacks the N-terminal part of the protein preceding the WD repeats motif, is unable to associate with RasSF1, although it includes the sequence 260-291.

This means that although this sequence 260-291 is essential for interaction with βTrCP, it is not sufficient and it requires to be fused to sequences at the N-terminal part of the protein.

### Example 11

1. ³⁵S Metabolic Labeling and Pulse-Chase Experiments.

24 h after electroporation with RasSF1 expressing plasmid and either the pAS1 B vector, or pAS1B HA-tagged βTrCP, or with pAS1B HA tagged βTrCPΔF, 10⁶ HeLa cells were incubated for 30 min in met-and cys-free medium (DMEM), then 125 μCi of [³⁵S]methionine-cysteine (NENlife) per ml were added to the same medium for 1 hour. Cells were washed in PBS and harvested (time 0) or incubated for 15, 30, 60, 120 min in complete medium (DMEM), washed again in PBS and lysed as described previously. Cell lysates were immunoprecipitated with 5 μg of mouse monoclonal anti-myc per ml (clone 9E10, SantaCruz) and incubated with protein G-Agarose (Sigma) beads. Beads were washed with lysis buffer supplemented with NaCl (300 mM final) and immune complexes were eluted with Laemmli sample buffer, separated on 12% SDS PAGE, fixed in acetic acid (10%)-methanol (30%), dried and exposed to Kodak X-OMAT film.

2. HA-βTrCP is co-immunoprecipitated with Myc-Ras SF1A in anti-Myc immunoprecipitate:

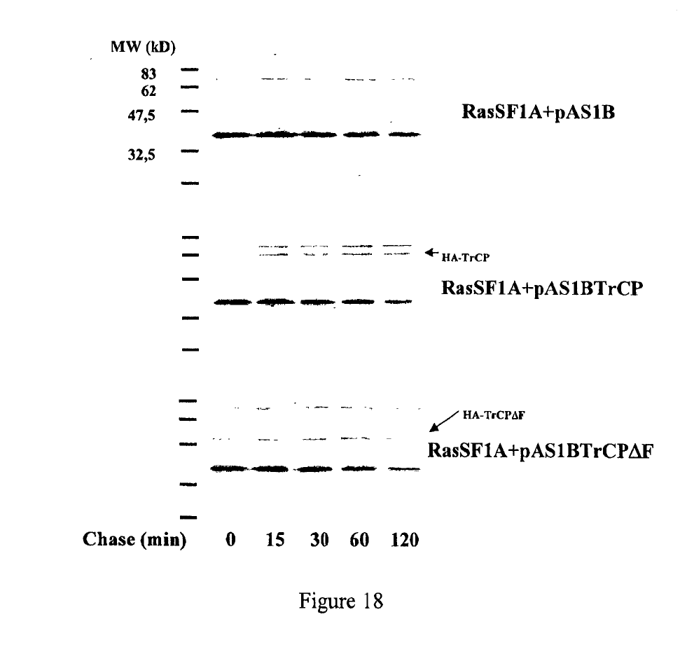

24 h after electroporation using Ras SF1A expressing plasmid and either the pAS1B vector (FIG. 18-upper panel), or pAS1B HA-βTrCP (FIG. 18-middle panel), or with pAS1B HA tagged βTrCPAF (FIG. 18-lower panel), HeLa cells were metabolically labeled with [³⁵S]methionine-cysteine for 1 hour. Cells were washed in PBS and harvested (time 0) or incubated for 15, 30, 60, 120 min in complete medium (DMEM), and lysed as described above. Cell lysates were immunoprecipitated with mouse monoclonal anti-myc (clone 9E10, SantaCruz) and incubated with protein G-Agarose (Roche) beads. Immune complexes were eluted with Laemmli sample buffer, separated on 12% SDS PAGE, fixed in acetic acid (10%)-methanol (30%), dried and exposed to Kodak X-OMAT film.

As shown in FIG. 18 HA-βTrCP (middle panel) or HA-βTrCPΔF (lower panel), are specifically co-immunoprecipitated with RasSF1A in the anti-myc immunoprecipitates. HA-βTrCP (middle panel) or HA-βTrCPΔF (lower panel) are identified by their respective molecular weight and their specific occurrence in the samples co-transfected with the corresponding plasmids. Neither of these bands are detectable in the sample co-transfected with the vector pAS1 B without insert (upper panel), which means that the immunoprecipitations are specific. These results confirm that either by immunoprecipitating RasSF1, or by immunoprecipitating βTrCP, one find the other partner co-immunoprecipitated in the immunoprecipitates. This is a strong confirmation that Ras SF1 and βTrCP are associated in human cells. From the point of view of protein stability, the expression of βTrCP or of βTrCPΔF does not seem to affect significantly the stability of RasSF1A, at least during the 120 min chase period.

### Example 12

1. Effect of Co-Transfected ds SF1 RNA on Myc-SF1 C and on β-catenin Expression:

This experiment was performed according to Elbashir et al. (2001). 200,000 Hela cells were co-transfected using lipofectamine (Invitrogen) with 0.5 μg of pMyc RasSF1C, together with 0, 0.1, 0.3, 1, 3, 10, or 30 nM RasSF1 dsRNA 185, or as indicated with both 15 nM of RasSF1 dsRNA185 and 15 nM of dsRNA202, both directed against RasSF1 sequence.

RasSF1 and Luc dsRNAs were obtained from Pharmacon Research Inc. Ras SF1 dsRNA185 sequences: sense strand: 5′GCU GAG AUU GAG CAG AAG AdTdT (SEQ ID No. 20) antisense strand: 3′dTdTCGA CUC UM CUC GUC UUC U5′ (SEQ ID No. 21) Ras SF1 mRNA sequence target of the dsRNA185: 5′AAG CUG AGA UUG AGC AGA AGA3′ (SEQ ID No. 22) Ras SF1 dsRNA202 sequences: sense strand: 5′GAU CAA GGA GUA CAA UGC CdTdT3′ (SEQ ID No. 23) antisense strand: 3′dTdTCUA GUU CCU CAU GUU ACG G5′ (SEQ ID No. 24) Ras SF1 mRNA sequence target of the dsRNA202: 5′MG AUC MG GAG UAC MU GCC3′ (SEQ ID No. 25)

Both the antisense and sense strands were annealed and then co-transfected with the pMyc RasSF1C or with the pAS1 B-βTrCP plasmids. As a control ds RNA Luc GL2 targeting the luciferase mRNA described in Elbashir et al.(2001) was used.

sense strand: 5′CGU ACG CGG MU ACU UCG AdTdT (SEQ ID No. 26)

antisense strand: 3′dTdTGCA UGC GCC UUA UGA AGC U5′ (SEQ ID No. 27)

After 24 h transfection, cells were washed in PBS and lysed in buffer (50 mM Tris HCl pH 7.5, 1 mM EDTA, 150 mM NaCl, 10% glycerol, 1% NP40, 1% antiprotease cocktail (Sigma), and analyzed by Western blot after SDS PAGE electrophoresis using anti-myc HRP (9E10, Roche), or anti-HA (3F10 high affinity, Roche) or anti-beta-catenin 2206, Sigma), or anti-tubulin ((TU-02) Santa-Cruz).

2. Inhibition of RasSF1 expression by RNAi targeting RasSF1 mRNA results in decrease of β-catenin expression, while overexpression of RasSF1C results in increase in \tilde{β}-catenin expression:

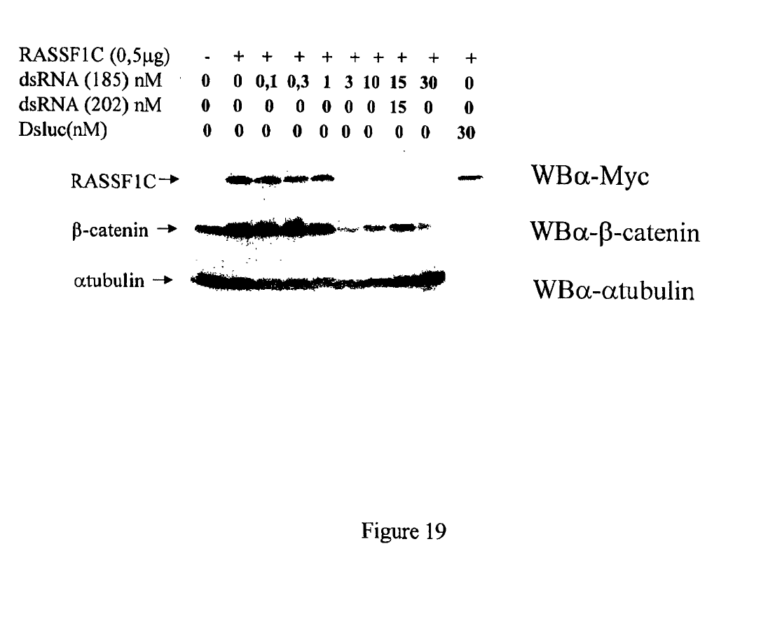

Hela cells were co-transfected using lipofectamine (Life echnology/Invitrogen) with transfected 0.5 μg of plasmid expressing Myc-Ras SF1 C together with various amount of double stranded small interfering RNA directed against SF1 mRNA coding for both the A and the C forms (dsRNA 185 or when indicated, a mixture of dsRNA185 and dsRNA202, both directed against the RasSF1 mRNA sequence), according to Elbashir et al. (2001). 24 h later these cells were lysed as described above and analyzed by Western blot for Myc-Ras SF1 C expression with anti-myc antibody 9E10 (Roche), or β-catenin expression with anti-β-catenin antibody (2206, Sigma), or with anti-tubulin expression using anti-tubulin antibody. As it can be seen in FIG. 19, transfection of dsRNA 185 leads to a gradual decrease of Myc-Ras SF1 expression. Myc-Ras SF1C became undetectable as soon as 3 nM or greater amounts (up to 30 nM). are co-transfected with RasSF1C. As a controls of specificity of RasSF1 dsRNA activity, one can see that the level of expression of Tubulin was not affected by dsRNA 185. Moreover, the effect of dsRNA on RasSF1C expression is sequence specific since dsRNA trageting luciferase had no effect on the expression level of Myc-Ras SF1C. Interestingly, while overexpression of RasSF1C without dsRNA resulted in increased level of β-catenin expression, the disappearance of Myc-Ras SF1C provoked by co-transfection with dsRNA resulted in decreased expression of β-catenin. Since β-catenin is a substrate of βTrCP, which controls its degradation, and since Ras SF1 interacts with βTrCP as shown here, these results favor the interpretation that RasSF1C acts as a negative modulator of β-TrCP activity.

Summary of the Results of βTrCP-RasSF1 Interaction with Different Constructs of βTrCP

Altogether, these results demonstrate that there is an association between Ras SF1 A or Ras SF1C in human cells, as well as in yeast by two-hybrid assay. The domain responsible for such interaction in the N-terminal half of βTrCP (see FIG. 20), between amino acids 260 to 291 was mapped. This domain is necessary but not sufficient. Its fusion with N-terminal sequences is also needed to get interaction of βTrCP with RasSF1.

Concerning Ras SF1, the amino acids 1 to 119 of RasSF1A or 1-49 of Ras SF1C are not needed for interaction with βTrCP. This interaction of RasSF1 with βTrCP is important since it was demonstrated that RasSF1 acts as a negative modulator of the activity of βTrCP in the control of expression and stability of an important substrate of βTrCP such as β-catenin. This interaction of RasSF1 with βTrCP could influence the activity of RasSF1 and its tumor suppressive functions in lung, breast and ovarian tumors. In particular the precise mapping of the interaction domains on both proteins could be used to modulate the function of RasSF1 in tumorigenesis in breast, lung, and ovarian tumors in which inactivation of RasSF1 has been associated with the cancer process (Dammann et al. 2000, Agathanggelou et al. 2000).

While the invention has been described in terms of the various preferred embodiments, the skilled artisan will appreciate that various modifications, substitutions, omissions and changes may be made without departing from the scope thereof. Accordingly, it is intended that the present invention be limited by the scope of the following claims, including equivalents thereof.

### REFERENCES

Margottin F., Bour S., Durand H, Selig, L., Benichou S., Richard V., Thomas D., Strebel K. and Benarous R.

A novel human WD protein, h-□-TrCP, that interacts with HIV-1Vpu connects CD4 to the ER degradation pathway through an F-box motif. Mol. Cell, (1998) 1, 565-574.

.Selig, L., Pages, J. -C., Tanchou, V., Preveral, S., Berlioz-Torrent, C., Liu, L. X., Erdtmann, L., Darlix, J. -L., Benarous, R. and S. Benichou.

Interaction with the p6 domain of the Gag precursor mediates incorporation into virions of Vpr and Vpx proteins from primate lentiviruses. J. Virol. (1999), 73, 592-600.

Lassot I., Segeral E., Berlioz-Torrent C., Durand H., Groussin L., Hai T., Benarous R. and Margottin-Goguet F.

ATF4 degradation relies on a phosphorylation-dependent interaction with the SCF-□TrCP ubiquitin ligase. Mol. Cell. Biol. (2001), 21, 2192-2202.

Dammann R., Li C., Yoon J. H., Chin P. L., Bates S. and Pfeifer G. P.

Epigenetic inactivation of a Ras association domain family protein from the lung tumor suppressor locus 3p21.3 Nature Genetics (2000), 25, 315319.

Morrissey C, Martinez A, Zatyka M, Agathanggelou A, Honorio S, Astuti D, Morgan N V, Moch H, Richards F M, Kishida T, Yao M, Schraml P, Latif F, Maher E R.

Epigenetic inactivation of the RASSF1A 3p21.3 tumor suppressor gene in both clear cell and papillary renal cell carcinoma. Cancer Res. 2001 October 1; 61 (19):7277-81.

Agathanggelou A, Honorio S, Macartney D P, Martinez A, Dallol A, Rader J, Fullwood P, Chauhan A, Walker R, Shaw J A, Hosoe S, Lerman M I, Minna J D, Maher E R, Latif F.

Methylation associated inactivation of RASSF1A from region 3p21.3 in lung, breast and ovarian tumours. Oncogene. 2001 March 22;20(12):1509-18.

Elbashir S M, Harborth J, Lendeckel W, Yalcin A, Weber K, Tuschl T. Duplexes of 21-nucleotide RNAs mediate RNA interference in cultured mammalian cells. Nature. 2001 May 24;411 (6836):494-8.

Hart M, Concordet J P, Lassot I, Albert I, del los Santos R, Durand H, Perret C, Rubinfeld B, Margottin F, Benarous R, Polakis P. “The F-box protein beta TrCP associates with phosphorylated beta-catenin and regulates its activity in cells,” Curr. Biol 1999 February 25; 9 (4):207-10 Related Articles, Books, Linkout.

What is claimed is:

1. A complex of protein-protein interactions as defined in SEQ ID Nos. 2 and 4.

2. A complex of polynucleotides as defined in SEQ ID Nos. 1 and 3 encoding for the polypeptides.

3. A recombinant host cell expressing the interacting polypeptides as defined in Table 1.

4. A method for selecting a modulating compound comprising:
(a) cultivating a recombinant host cell with a modulating compound on a selective medium and a reporter gene the expression of which is toxic for said recombinant host cell wherein said recombinant host cell is transformed with two vectors:
(i) wherein said first vector comprises a polynucleotide of SEQ ID No. 1 encoding a first hybrid polypeptide and a DNA bonding domain;
(ii) wherein said second vector comprises a polynucleotide of SEQ ID No. 3 encoding a second hybrid polypeptide and an activating domain that activates said toxic reporter gene when the first and second hybrid polypeptides interact;
(b) selecting said modulating compound which inhibits the growth of said recombinant host cell.

5. A modulating compound obtained by the method of claim 4.

6. A vector comprising the polynucleotide comprising the SEQ ID Nos. 1 or 3.

7. A fragment of a polypeptide comprising SEQ ID Nos. 2 and 4.

8. A variant of a polypeptide comprising SEQ ID Nos. 2 and 4.

9. A fragment of a polynucleotide comprising SEQ ID Nos. 1 and 3.

10. A variant of said polynucleotide comprising SEQ ID Nos. 1 and 3.

11. A recombinant host cell containing the vectors according to claim 6.

12. A pharmaceutical composition comprising a modulating compound of claim 5 and a pharmaceutically acceptable carrier.

13. A pharmaceutical composition comprising the recombinant host cells of claim 11 and a pharmaceutically acceptable carrier.

14. A protein chip comprising the polypeptides of claim 7 or 8.

15. A monoclonal antibody of the protein complex of claim 1.
